(19) United States
(12) Patent Application Publication
(10) Pub. No.: US 2011/0313344 A1
(43) Pub. Date: Dec. 22, 2011

(54) Method for treating refractive errors and vision disorders of an eye

(76) Inventor: Albert Daxer, Linz (AT)

(21) Appl. No.: 12/932,347

(22) Filed: Feb. 24, 2011

Related U.S. Application Data
(63) Continuation-in-part of application No. 10/555,353, filed on Sep. 29, 2006, now Pat. No. 7,922,735.
Application No. 10/555,353 is a continuation-in-part of application No. 12/224,966, filed on Sep. 10, 2008.
Application No. 12/224,966 is a continuation-in-part of application No. 12/227,533, filed on Nov. 20, 2008.
Application No. 12/227,533 is a continuation-in-part of application No. 12/925,162, filed on Oct. 14, 2010.
(60) Provisional application No. 61/279,824, filed on Oct. 26, 2009.

(30) Foreign Application Priority Data
May 2, 2003 (AT) A 663/2003
May 30, 2003 (AT) A 840/2003
Aug. 12, 2003 (AT) A 1264/2003
Feb. 25, 2004 (AT) A 299/2004
Mar. 16, 2006 (AT) A 428/2006
May 23, 2006 (AT) A 885/2006

(51) Int. Cl. A61F 9/013; A61M 37/00; A61B 18/18; A61F 9/007

(52) U.S. Cl. 604/22; 606/166; 606/107; 606/33

(57) ABSTRACT

A method for treating refractive errors and vision disorders of an eye includes fixing a suction ring to an eye, applanating a cornea of the eye, guiding a blade into the cornea with a guiding mechanism, creating a pocket inside the cornea by guiding the blade inside the cornea, removing the blade from the cornea and removing the suction ring from the eye.

### CROSS REFERENCE TO RELATED APPLICATIONS

This application is a continuation-in-part of U.S. application Ser. No. 10/555,353 filed Sep. 29, 2006, which application claims priority under 35 U.S.C. §119 of Austrian Patent Application Serial No. A663/2003 filed May 2, 2003, Austrian Patent Application Serial No. A840/2003 filed May 30, 2003, Austrian Patent Application Serial No. A1264/2003 filed Aug. 12, 2003 and Austrian Patent Application Serial No. A299/2004 filed Feb. 25, 2004 and which application also claims priority under 35 U.S.C. §365 of PCT/AT2004/000147 filed Apr. 30, 2004. The international application under PCT article 21(2) was not published in English.

This application is also a continuation-in-part of U.S. application Ser. No. 12/224,966 filed Sep. 10, 2008, which application is the National Stage of PCT/AT2007/000130 filed on Mar. 16, 2007, which claims priority under 35 U.S.C. §119 of Austrian Application No. A 428/2006 filed on Mar. 16, 2006. The international application under PCT article 21(2) was not published in English.

This application is also a continuation-in-part of U.S. application Ser. No. 12/227,533 filed Nov. 20, 2008, which application is the National Stage of PCT/EP2007/055015 filed on May 23, 2007, which claims priority under 35 U.S.C. §119 of Austrian Application No. A 885/2006 filed on May 23, 2006. The international application under PCT article 21(2) was not published in English.

This application is also a continuation-in-part of U.S. application Ser. No. 12/925,162 filed Oct. 14, 2010, which application claims priority under 35 U.S.C. §119(e)(i) and the benefit of U.S. Provisional Application No. 61/279,824 entitled “Method to treat corneal diseases such as keratoconus” filed on Oct. 26, 2009, which is incorporated in its entirety by reference herein.

### BACKGROUND OF THE INVENTION

1. Field of the Invention

The invention relates to a device for cutting the cornea of an eye in order to correct the refractive power thereof, having a frame that comprises a fixation ring, which may be drawn onto the eye, as well as a receptacle, which may be coaxially displaced relative to the fixation ring and which serves to accommodate an applanator for deforming the cornea within the fixation ring, and a holding device, which is guided on the frame in a plane that is perpendicular to the axis with regard to the fixation ring and which serves to hold a blade that passes through the frame via a peripheral recess and is mounted in front of the applanator, being radially displaceable relative to the fixation ring as well as movable around an axis perpendicular to the guiding plane via the holding device, for the purpose of cutting a pocket through a merely tunnel-like entry into the corneal tissue.

The invention further relates to an annular cornea implant for inserting into a cornea pocket of the human eye via a narrow, preferably tunnel-shaped access, with the end shape of the cornea implant depending on the refractive error to be corrected.

The invention further relates to a corneal implant to be inserted into the optical center of the cornea of the human eye for the purpose of correcting impaired vision, in particular presbyopia in otherwise emmetropic eyes (eyes with normal vision) as well as presbyopia in combination with hypermetropia (farsightedness) or myopia (nearsightedness).

The invention further relates to a procedure for correcting impaired vision in the human eye, in particular for correcting presbyopia, presbyopia in combination with hypermetropia, presbyopia in combination with myopia, and presbyopia in combination with astigmatism, by inserting a corneal implant into the optical center of the cornea.

The invention further relates to methods for treating corneal disease. In particular, the invention relates to methods for treating corneal disease, such as keratoconus, by forming a corneal pocket in the cornea, introducing a corneal stiffening substance into the corneal pocket and irradiating the cornea with electromagnetic radiation.

The invention further relates to method for treating refractive errors and vision disorders of an eye. In particular, the invention relates to methods for treating refractive errors and vision disorders of the eye by fixing a suction ring to an eye, applanating a cornea of the eye, guiding a blade into the cornea with a guiding mechanism, creating a pocket inside the cornea by guiding the blade inside the cornea, removing the blade from the cornea and removing the suction ring from the eye.

2. The Prior Art

In order to be able to correct the refractive power of the cornea of an eye, a known method (US 2001/0004702 A1) is to treat the interior of the corneal tissue via an entry that may, for example, be approximately 4 mm wide. This method serves to create a pocket inside the corneal tissue, into which an implant for refractive power correction may be inserted. In comparison to other methods for correcting the refractive power, where either the surface of the corneal tissue is ablated (DE 34 33 581 A1) or the cornea is broadly incised to produce a flap that may be folded back (LASIK method), treating the interior of the cornea via a tunnel-like entry has the advantage that postoperative pain and/or impairment of the stability of the cornea are kept to a minimum since the top layer essential for the stability of the cornea, which also includes the Bowman's membrane, remains largely uninjured.

Therefore, treating the cornea via a tunnel-like entry offers multiple advantages in relation to the other methods of treatment. In order to be able to perform such a refractive power correction, US 2001/0004702 A1 suggests attaching a frame to the eye of the patient on which a holding device for a movable blade is mounted. The blade passes through the frame via a peripheral recess and is guided in a guiding plane by means of a holding device. This guiding plane lies parallel to the cutting plane of the blade, a pocket being produced in the cornea in said cutting plane. The blade in its cutting plane is additionally held down by a spring so as to rest on the frame as slipping of the blade when being placed on the cornea for producing the tunnel-like entry must be avoided. That is to say, it is relatively difficult to penetrate the elastic and extremely tough outer layer of the cornea. Moreover, the blade must not get deformed during cutting as in that case an exact cutting plane and prevention of perforation of the outer or inner layer of the cornea may not be guaranteed, whereby the successful correction of the refractive power would be precluded. While this eventuality may be counteracted by using blades of greater hardness (e.g. made of diamond material), such blades are particularly sensitive to shear stress, which excludes the use of said blades in the device according to US 2001/0004702, given the fact that the blade rests on the frame. Moreover, such blades bear the risk of breakage and also their sharpness would be greatly impaired.

Another known state-of-the-art method (LASIK, U.S. Pat. No. 5,556,406 A) consists in broadly incising the corneal tissue to produce a flap which is then folded back, and ablating a portion of the exposed corneal tissue by using a laser, or alternatively applying an artificial lens before placing the corneal flap back in its original position. This method has the disadvantage that, as already noted, the stability of the cornea is greatly impaired, which may occasionally result in undesirable bulges of the corneal surface.

The optical apparatus of the human eye for depicting the environment basically consists of the cornea and the lens which is positioned behind the iris. This optical apparatus of the eye has a total refractive power of approximately 60 dioptres, with the interface between the cornea and the air—i.e. the outer boundary of the eye—with approximately 40 dioptres accounting for most of the refractive power. This refractive power of the cornea is basically indirectly proportionate to the radius of the cornea surface (interface between cornea and air). A change in the radius of the curvature of the cornea also leads to a change in the refractive power of the eye. By increasing the (central) radius of curvature of the cornea the refractive power decreases, a fact which the eye surgeon takes advantage of in refractive surgery for the correction of shortsightedness. The refractive surgical techniques using laser (LASIK, LASEK, etc.) remove more corneal tissue from the central parts than from the peripheral parts of the cornea.

With the LASIK technique, for instance, a lamellar “flap” is cut into the cornea at a certain depth. Such flaps have the major disadvantage that they significantly impair the biomechanical stability of the cornea. The flap, in particular, no longer fully adheres to the underlying corneal tissue. This reduces the bio-mechanically effective cross section of the cornea by exactly the amount that corresponds to the thickness of the flap. The preferable option would be to undertake corrective measures on the inside of the cornea, without risking such a massive biomechanical impairment as is caused by a flap. Moreover, the laser techniques mentioned above are only suited for the treatment of myopic patients with up to 10 dioptres.

To avoid these drawbacks, methods have been developed in which cornea implants are used that cause a deformation of the cornea to the effect that the radius of curvature of the cornea is enhanced by adding volume, which in turn reduces the refractive power and thus corrects the myopic eye.

Cornea implants are mostly annular or ring-shaped, using either full rings (open or closed) or parted rings (e.g. ring segments).

US 2005/0119738 A1, for example, reports the use of such a cornea implant underneath a flap. It describes an annular implant for insertion into the cornea underneath a flap. In this case, a central part of the cornea implant is directly optically effective, i.e. in order to fulfill its task, it must be part of the optical zone and be penetrated by the rays of light to be depicted. The cornea implant has the task to form a multifocal corneal surface, with the area of the central hole in the cornea implant being responsible for near vision and the area that also includes the inner part of the cornea implant (optical zone) for far vision. To be able to solve this difficult task, the optical zone (including the inner part of the cornea implant) must be located within the diameter of the pupil of the eye, which is mostly 2 to 5 mm. The inner hole of the cornea implant therefore must not be essentially larger than 2 mm. Moreover, the inner part of the cornea implant, which is intended to be directly involved in producing an image in far vision, must have a minimum width which, taking into account the optical laws, must substantially exceed 1 mm (2 mm) in order to be effective. Added to this is the outer part of the cornea implant. This results in the substantial problem that the significant material width, which by far exceeds 1 mm, may lead to problems with oxygen and nutrient supply. While the problem with nutrient supply may be ameliorated by using the microporous hydrogel indicated, problems with stability and stabilization of the ring geometry still tend to arise due to the softness of the ring.

Moreover, the cornea implant has no pointed end on its inner or outer edge, but a finite edge thickness of preferably 10 μm. These edges have the considerable disadvantage that deposits may occur in the area, leading to vision impairment.

Experience with multifocal imaging, both in contact lens adjustment and in the implantation of intraocular lenses in cataract surgery, shows that this is tolerated by only a small minority of patients. The majority of patients perceive the simultaneous “blur” of the focal point along the optical axis as disturbing rather than beneficial. Moreover, it is to be expected that with respect to the present structure, patients will perceive the central part of the cornea implant, and here in particular the inner edge that needs to lie within the pupil width, as disturbing (despite adjustment to the refractive index of the surrounding medium).

As can be seen, the insertion of a cornea implant into the cornea underneath a flap has considerable drawbacks and in particular results in a significant impairment of the stability of the cornea on account of the lamellar cut in the cornea for producing a flap. This impairment of the stability of the cornea is typically experienced in LASIK surgery, where a similar flap needs to be created.

Ring-shaped cornea implants are also used in conjunction with techniques envisaging a circular incision in the corneal surface. GB 2095119 A, for instance, describes such a circular incision from the corneal surface into the corneal tissue, into which a ring of approximately 8 mm in diameter is introduced for the purpose of flattening the central cornea, which leads to myopia correction. Two different ring geometries are described and reference is made to their size. A circular ring section with a thickness of approximately to 0.5 mm and a triangular ring section with an edge length of approximately 0.3 mm are described. The materials mentioned are essentially polymer plastics. The disadvantage of this method is the significant traumatization of the tissue. After inserting the ring into the corneal tissue, for instance, the incision needs to be closed with a suture along the full length of the circumference as otherwise the ring could not be maintained in a stable position inside the cornea, and the stability of the cornea would be massively impaired. Without a suture, the extent of correction, i.e. the dioptres treated, would not be controllable. But even with a suture, the extent of correction can only be foreseen to a certain extent if the ring is rigid enough and only marginally deformable to withstand a potential pull of the tissue resulting from the suture.

WO 93/12735 A1 describes a variant of GB 2095119 A with a bio-compatible, annular cornea implant, which is also inserted into the corneal stroma through a circular incision in the corneal surface for the purpose of correcting the refractive power in the myopic eye. Said implant is a ring with a fixed, i.e. unchangeable diameter, a refractive index that does not deviate from that of the corneal tissue by more than 2% and may have the following dimensions: ring diameter of approximately 2.4 mm to 12 mm, ring width of approximately 0.2 mm to 4 mm and ring thickness of approximately 0.005 mm to 0.2 mm. The ring has a convexly shaped front side and a flat back side. In this case, too, the traumatization of the tissue when inserting the implant is considerable. Another major drawback when using the implant inside the cornea is the flat back side of the ring. Since the corneal surface is curved with a radius of approximately 8 mm, this leads to the fact that with the ring widths indicated, the shape of the back side of the ring and the corresponding corneal cut inside the cornea pocket are in part considerably different from each other. Such a disproportionate geometry may, as frequently described in the literature, lead to massive accumulation of deposits of organic material along the interfaces, which may lead to vision impairment and undesirable cosmetic results. Moreover, this results in a highly uneven distribution of pressure on the tissue across the back face of the ring, which may induce pressure atrophies and tissue necrosis.

To avoid the disadvantages described above, methods have been developed where the respective cornea implant is inserted into a fully enclosed cornea pocket via a narrow, tunnel-shaped access. Since the inner tension of the cornea acts alongside the'corneal lamellae, the formation of a basically enclosed cornea pocket does not reduce the bio-mechanically effective cross section for this tension and the bio-mechanical stability of the cornea is not impaired.

A suitable procedure and a suitable device for creating such a cornea pocket is, for instance, described in EP 1 620 049 B1, the disclosure of which is hereby incorporated herein by reference.

The creation of such a cornea pocket including a narrow, more or less tunnel-shaped access is, however, already known from US 2002/0055753 A1. After its insertion into the cornea pocket, the folded annular cornea implant is unfolded and placed in position. Although the insertion via a narrow tunnel works really well with these highly flexible, foldable cornea implants, this well-known procedure and the cornea implants inserted therewith have the decisive disadvantage that said implant has to be manually unfolded inside the pocket after insertion. However, a multitude of forces are effective inside the pocket after insertion of the cornea implant which prevent the cornea implant from unfolding itself, so that especially in the case of very flexible rings, there is a risk that the cornea implant does not resume its original predefined shape after implantation and astigmatisms or higher-order aberrations may be induced. Tedious, complicated manual manipulations therefore need to be taken into account after implantation to restore the initial ring form. But resuming the exact shape is often impossible and so the desirable correction of the refractive error may not be achieved. Moreover, the need for the cornea implant to be manually unfolded in the cornea pocket puts the cornea itself and above all the narrow, preferably tunnel-shaped access into the cornea pocket under unnecessary strain. In other words, a sufficiently flexible state-of-the-art cornea implant may facilitate the insertion into the cornea pocket via the narrow access, but simultaneously complicates the unfolding inside the cornea pocket.

An object of the present invention therefore is to provide a cornea implant which has none of the drawbacks described above and may be used in a vision correction procedure, where a cornea implant is inserted into a cornea pocket via a narrow, preferably tunnel-shaped access.

As set forth above, the optical apparatus of the human eye that generates an optical image of the environment basically comprises the cornea and the lens, which is positioned behind the iris. This optical apparatus of the eye has a total refractive power of approximately 60 dioptres, with the interface between the cornea and the air—i.e. the outer boundary of the eye—with approximately 40 dioptres accounting for most of the refractive power. This refractive power of the cornea is in general indirectly proportionate to the radius of the corneal surface (interface between cornea and air). A change in the radius of the curvature of the cornea also leads to a change in the refractive power of the eye.

In the case of myopia or nearsightedness, the eyeball is too long and the refractive power of the cornea thus inadequate to assure that the light rays are focused on the retina; these are focused in front of the retina instead.

In the case of hypermetropia or farsightedness, the eyeball is too short and the refractive power of the cornea thus insufficient to assure that the light rays are correctly focused on the retina; they are focused behind the retina instead.

Presbyopia is a dissociation of the refractive power of the eye in that for accurate far vision a different correction of dioptres than for accurate near vision is required.

Different options for correcting these refractive errors are available. In addition to the classical methods of vision correction via glasses or contact lenses, surgical methods are also known wherein implants are inserted into the cornea of the human eye with the aim to either modify the curvature of the cornea and thus correct the refractive power of the latter accordingly, or to alter the optical properties of the cornea through the optical properties of the implant.

By enlarging the radius of curvature of the cornea the refractive power is reduced, which allows to correct a myopic condition. To be able to correct a hyperopic condition, the corneal radius needs to be reduced, i.e. the curvature needs to be increased.

To be able to correct presbyopia by surgical intervention, it is necessary to impart a certain degree of bi-focality or multi-focality to the refractive power of the cornea. This means that the refractive power of the cornea is designed in a way that the light rays entering the eye from different distances (near or far away), depending on their point of entry, are simultaneously focused on the retina, or more precisely in the central area of the retina (i.e., the macula, the area where accurate vision occurs). This implies that one or several images from a far distance and one or several images from a near distance are simultaneously focused in the macula. The brain then selects the appropriate image. To allow this selection to take place, the far-away image and the near image must have about the same intensity. The use of some kinds of contact lenses and intraocular lenses, which are inserted after cataract surgery, are based on this principle.

WO 93/05731 relates to the insertion of an optical lens into the optical center of the cornea, the dimensions of which are smaller than those of the optical zone being limited by the diameter of the pupil.

The optical center of the cornea is that part of the cornea along which the optical axis of the eye passes through the cornea. The optical axis is the axis of projection of the optical system of the eye. The ophthalmologist determines the optical center by using specific assessment methods. The ophthalmologist may choose from a wide variety of different methods. The methods for determining the optical center of the cornea described hereinafter represent only a small portion of the many different methods commonly applied, and are not exhaustive. Many systems, in particular excimer laser systems with active eye tracking, use the center of the pupil or its projection on the corneal surface or around a point at an individually defined distance as the optical center of the cornea. Other common systems are aimed at the area where the curvature of the cornea is most pronounced. Especially in the case of high-degree myopia, in fact, an angular deflection of the optical axis from the anatomical axis is to be noticed, which is defined as the “kappa angle”. Another method relates to the so-called “Purkinje reflexes”. These are reflexes on the corneal front and back faces as well as on the lens front and back faces, which occur when the patient focuses on a preferably point-shaped light source. Although these reflexes ideally overlap, most of the time this is not the case; the eye specialist then chooses one of these reflexes as the optical center. It is also quite common to choose the middle position of all four reflexes, or the middle between this middle position and the center of the pupil, etc. Eventually, it is left to the personal discretion, individual experience and preference of the eye specialist how he determines the optical center of the cornea. Generally speaking, the various methods used for determining the optical center of the cornea tend to render quite similar results.

In WO 93/05731, the implantation of an optical lens in the optical center of the cornea results in various zones of different refractive power, namely in the area of the optical lens itself as well as in the adjoining corneal tissue through the refractive power of the cornea proper. This allows the creation of a certain degree of bifocality or multifocality, depending on the contour of the optical surface of the implanted lens. The thickness of the lenses in the direction of the optical axis of the eye is less than 50 μm to avoid an undesirable deflection of the cornea and an impairment of the refractive power of the lens. Basically, however, there is the disadvantage that the newly created boundary surfaces may produce optically adverse phenomena such as glares and reflections, which the patient will find disturbing. The optical surface therefore needs to be excellently designed, which in case of such small dimensions is a rather difficult and tedious undertaking. It is also known that organic deposits tend to form along the boundaries of corneal implants, which may substantially impair the function of the implants as optical elements.

U.S. Pat. No. 6,589,280 B1 describes a method of creating a multi-focal cornea by implanting a minimum of 50 microscopically small optical lenses outside the optical center of the cornea. Each lens should have a defined refractive power, preferably 1 to 3 dioptres. The optical lenses have a thickness of approx. 2-3 microns and a width of less than 1 mm (measured in a plane perpendicular to the direction of thickness). The lenses are so small that the curvature of the cornea is not impaired by the deflection of the corneal surface. The refractive error is corrected exclusively through the different refractive power of the individual lenses. The described method is extremely complicated and, with regard to its usability in living tissue, the same arguments as mentioned earlier apply.

U.S. Pat. No. 5,722,971 describes a method wherein a thin plate-shaped implant with diffractive optics and a hole at its center is implanted. The outer diameter is in a range between 3 mm and 9 mm. In addition, ring implants as well as ring replacement implants are presented. In this case, the ring is replaced by several individual implants which are concentrically positioned along a circle around the center of the cornea. By leaving individual positions of the circle empty, not only myopic conditions but also regular and irregular astigmatisms may be corrected. No reference is made to the dimensions of the replacement implants, but the illustrations provided reveal that in order for the replacement implants to have the same effect as the rings, they must replace about the same volume and therefore, as is also shown in the drawings, must have a much bigger size and dimensions that correspond to the pupil width or iris width. Moreover, there is no detailed information as to their geometry. The illustrated applications imply that they must have the shape of a protracted ellipsoid. Such implants are not suited for use in the area of the central cornea.

The same applies with respect to US 2004/0073303 A1, wherein the preferred embodiment of the invention is even a curved, protracted implant (centroid).

A state-of-the-art method therefore is to implant optical lenses as corneal implants in the optical center of the cornea. These optical corneal implants exert their effect via their own refractive power. They have an optically effective front and/or back face and also contain a material with a specific refractive index, which is positioned between the optically effective front and/or back face and defines both the contour and the refractive power of these optical corneal implants. It is also known, however, that in such optical corneal implants there is the tendency that in the area of contact with the surrounding tissue purely optical phenomena occur and organic material is deposited. Especially in implants which are inserted into the optical center of the human eye, this leads to a significant impairment of vision.

Although implants positioned outside of the optical center are less sensitive to the aforementioned deposits, they are not able to create bi-focality, let alone multi-focality to correct presbyopia on its own or in combination with hypermetropia or myopia.

The cornea is the transparent, projecting outer “wall” of the eye. The optical quality of the cornea is of particular importance for the visual function of the eye. The outer corneal surface, which forms the interface between the cornea and the surrounding air, is responsible for approximately two thirds of the total dioptic power of the eye. Accordingly, a regular shape of the cornea is required to achieve good quality of vision.

There are several corneal diseases affecting the regularity of the cornea and reducing the visual function of the eye. Among these diseases, Keratoconus is one of the most prominent. The majority of these corneal diseases exhibit a progressive character, wherein the degree of corneal irregularity and visual dysfunction increases over time.

Seiler and his coworkers have developed a method of corneal cross-linking for reducing the irregularity of the cornea and stopping the progression of the disease, as described, for example, in Spörl E, Huhle M, Kasper M, Seiler T. (1997) Erhöhung der Festigkeit der Hornhaut durch Vernetzung. Ophthalmologe; and in Schnitzler E, Spörl E, Seiler T. (2000) Bestrahlung der Hornhaut mit UV-Licht and Riboflavingabe als neuer Behandlungsversuch bei einschmelzenden Hornhautprozessen, erste Ergebnisse bei 4 Patienten. Klin Monatsblatt Augenheilkunde. This therapeutic procedure of corneal cross-linking includes the removal of the epithelium of the cornea and the application of eye drops of riboflavin at the de-epithelialized corneal surface for thirty minutes and consecutive ultraviolet (UV) irradiation of the cornea for another thirty minutes.

The application of riboflavin eye drops and UV irradiation without epithelial removal was shown to be clinically ineffective. The removal of the epithelium, however, results in severe pain and photophobia for several days after surgery. Moreover, although the known method is effective in stopping the progression of the corneal disease, the ability of the known method to cure the irregularity of the diseased cornea is very limited.

Accordingly, there exists a need for an effective method for treating corneal disease which preserves the epithelium. Moreover, a need exists for a painless method for stopping the progression of corneal disease which method provides the option of effectively correcting even higher grades of corneal irregularities than can be corrected using prior known methods.

### SUMMARY OF THE INVENTION

An underlying objective of an aspect of the present invention therefore is to provide a method for cutting the cornea of an eye to correct its refractive power on the basis of the state-of-the-art technique initially described in such a way that, departing from the very point at which the tip of the blade is placed on the cornea, a pocket lying in the cutting plane of the blade may be cut via a tunnel-like entry, without any impairment of the stability of the cornea to be expected. In addition, the device shall be aimed at allowing a broad range of refractive power corrections.

An aspect of the invention may accomplish this objective in that the blade passes through the frame recess with clearance, and that the holding device supports a vibrator for setting the blade in oscillatory motion in the cutting plane. Having the blade pass through the frame recess with clearance ensures that the blade does not rest on the frame during displacement, particularly when introducing the blade into its cutting position, so that damage to the blade and/or the edge of the blade may be precluded. It is therefore possible to use comparatively hard materials for the blade since the brittleness of these usually very hard materials need not be taken into account. In contrast to US 2001/0004702 A1, therefore, no breakage and/or impairment of the edge of a blade is to be expected as the blade is guided without its sensitive parts being in contact with the frame, so that according to an aspect of the invention blades with a greater cutting capability and smaller dimensions may be used.

Especially when penetrating the outer layer of the cornea to prepare a tunnel-like entry, it may thus be assumed that the tunnel-like entry most accurately adjoins the point at which the tip of the blade was previously placed on the cornea. The penetration of the blade is further facilitated by having the holding device support a vibrator for setting the blade in oscillatory motion in the cutting plane. This in particular serves to overcome the elasticity of the outer layer of the cornea without risking an enhanced indentation of the corneal surface. The vibrator also ensures that minimal force is exerted during the cutting process, so that any movement of tissue, which is attributable to the motion of the blade resulting from the elasticity and toughness of the tissue and which tends to arise even when using extremely sharp blades, can be precluded. In comparison to the other state-of-the-art methods, thus a specially drawn cut for high cutting precision may be ensured.

If the receptacle is designed as a receptacle for the stop-delimited receipt of interchangeable applanators with differently curved contact faces intended for applanation of the cornea, said contact faces in sequential incisions defining pockets for delimiting a lens-shaped portion of tissue, it is possible to cut out a portion of tissue via a tunnel-like entry without the need to produce a corneal flap. The lens-shaped portion of tissue which is cut out may subsequently be pulled out via the tunnel-like entry, so that it becomes possible according to the invention to produce a defined cavity in the cornea, thereby insignificantly impairing the stability of the cornea in comparison to the other methods. This allows the creation of a tissue lens inside the cornea through which a refractive error may be corrected.

If the peripheral edges of the contact faces of the applanators inserted in the receptacle one after another have a different perpendicular distance from the cutting plane of the blade, the cutout of a lens-shaped portion of tissue is thus facilitated. While said difference of the perpendicular distances requires that two tunnel-like entries be produced in the corneal tissue, this has the advantage that the two pockets are prominent in their shared sectional line, which excludes the possibility that the portion of tissue is cut out incompletely.

If the non-metallic blade forms a sharp tip, the blade can penetrate more easily into the tissue from where it is placed on the cornea. If the blade additionally has a sharp edge extending towards the tip, a particularly narrow tunnel-like entry may be produced by radially displacing the blade relative to the fixation ring, which provides beneficial results for the stability of the cornea.

Particularly advantageous cutting properties of the blade are obtained by designing the blade as a double-edged knife, which is preferably made of diamond material, the blade having a maximum width of 2 mm, a maximum thickness of 200 microns and a blade length of at least 8 mm, preferably 10 mm.

If the applanator is made of transparent material, it becomes easy for the surgeon to monitor the contact face and/or the cutting process. If the applanator is additionally designed as an enlargement lens, the focal point of which lies in the area of the contact face for applanation of the cornea, preferably on the axis of symmetry of the applanator, the monitoring process is even further facilitated.

In order to be able to optimally adjust the size of the contact face of the applanator placed on the eye and/or obtain as exact a cutting area of the pocket as possible, it is recommendable to provide the transparent applanator with markings on its side facing the eye for determining the size of the contact face of the applanator as well as the cutting face on the eye.

If the option of using interchangeable applanators for cutting out a lens-shaped portion of tissue is to be dispensed with, this may be achieved by at least making the applanator contact face of a deformable material, which may then be manipulated by means of an actuator to obtain different curvatures. The actuator thus serves to predefine contact faces with different curvatures for the applanator inserted in the receptacle so as to ensure that a lens-shaped portion of tissue is cut out.

If the blade holder is equipped with a position adjuster operating perpendicular to the cutting plane of the blade, it becomes easy for the surgeon to determine at what depth from the corneal surface to cut the pocket as said position adjuster serves to adjust the distance of the blade from the applanator and/or the contact face of the applanator on the eye.

The blade holder consists, for example, of a lever system comprising at least two lever arms with pivot axes which are perpendicular to the cutting plane of the blade. If one arm receives the blade and the other arm is linked to the frame, this provides a simple construction ensuring that the blade can be radially displaced relative to the fixation ring as well as moved around the axis vertical to its guiding plane.

A further option is for the blade holder to comprise a forked blade guide, which is guided, possibly without clearance, between parallel faces of a peripheral groove provided on the frame, in particular on the receptacle. Not only is this embodiment exceptionally simple in its construction, it also predefines the space within which the surgeon may move the blade.

In order to render the insertion and/or interchange of applanators with their differently curved contact faces as simple as possible, it is suggested that the applanator be held in the receptacle in a stop-delimited position by using a partial vacuum. The only prerequisite for this purpose is to furnish the receptacle with a pressure line which draws out the air between the recess and the applanator.

In a further aspect of the invention, an annular cornea implant for inserting into a cornea pocket of the human eye via a narrow, preferably tunnel-shaped access, with the end shape of the cornea implant depending on the refractive error to be corrected, has a shape memory which is impressed on the basis of the geometry and/or material of the implant, and is designed in such a way that the deformability from a starting shape enables the insertion of the cornea implant into the cornea pocket via the tunnel-shaped access and the cornea implant has an adjustment force in an end shape thereof, which enables an essentially independent unfolding of the cornea implant in the cornea pocket.

According to one embodiment of the invention, the starting shape and the end shape are identical.

To avoid an impairment of the biomechanical stability of the cornea as results when cutting a flap, the width of such a tunnel-shaped access to an otherwise closed cornea pocket should typically not exceed 5 mm, and should ideally lie between 2 mm and 3 mm. A cornea implant according to the invention may therefore be inserted into the cornea pocket via a narrow access, the maximum width of which is below 5 mm, and preferably between 2 mm and 3 mm, to avoid an impairment of the biomechanical stability of the cornea as results when cutting a flap, without breaking or being altered in its shape (such as by permanent (irreversible) plastic deformation).

At the same time it is assured that the cornea implant, in addition to being sufficiently deformable, also has the ability to create an adequate adjustment force so as to safely unfold into a predefined form—its end shape—inside the cornea pocket after implantation. It unfolds more or less independently and automatically, without any additional or essential, additional manual interference.

A precondition for this is that on account of the impressed shape memory, one or several optional end shapes may be pre-programmed in the cornea implant according to an aspect of the invention, and the latter, being either plastically or elastically deformed, assumes one of these end shapes either automatically or through activation by a trigger signal.

The shape memory is either impressed on the basis of a suitable chosen material, or on the basis of a special geometry of the cornea implant, or on the basis of a material-geometry combination.

The materials suited for insertion may be elastically or non-elastically deformable (plastic) materials. In elastic materials with a shape memory, the deformability and adjustment force mainly results from the elasticity of the material, such as PMMA (polymethyl methacrylate), silicone, etc. In non-elastic materials such as shape memory alloys, the adjustment force results, for example, from atomic forces which are released when one grid structure is spontaneously transformed into another.

Basically, there is a multitude of materials with a shape memory, which facilitate a shape memory of the cornea implants according to aspects of the invention. These materials include PMMA, polymers of EEMA or HEMA, or other acrylic materials, hydrogels, nylon, polycarbonate, polyethylene or other plastics, plastics with a temperature-dependent shape memory, shape memory alloys (e.g. based on Ni—Ti, Cu—Zn—Al, Cu—Al—Ni, etc.), suitable compounds of plastics and metals or non-metals (e.g. ceramics, semi-conductors, etc.), suitable compounds of metals and non-metals (e.g. ceramics, semi-conductors, etc.), or composite materials. Some of these materials, such as hydrogels, have already been used in cornea implants, but only the conditions under which they are applied and in particular the geometry of the cornea implant lead to the impression of the desirable shape memory.

It is important to assure that the desirable end shape can be achieved, by means of the adjustment force, from the intermediary shape into which the cornea implant needs to be deformed in order to be able to be inserted into the cornea pocket via the narrow access, irrespective of whether the starting shape and the end shape are identical or not.

The task according to the invention may, as mentioned before, also be solved exclusively or additionally by envisaging a special geometry of the cornea implant.

Therefore known elastic materials which receive the desirable shape memory due to their geometrical design may also be used as a cornea implantation body.

An exact fitting of the adjustment force is of vital importance because the cornea implant is only in its end shape suited to correct the respective refractive error. If the end shape cannot be achieved or is not exactly achieved, the correction of the refractive error is also inadequate.

After being implanted into the cornea pocket, the cornea implant is exposed to considerable forces that act against its unfolding, such as the frictional forces resulting from the pocket walls between which the cornea implant is positioned. This is further enhanced by forces resulting from the intrinsic tension of the cornea and the intraocular pressure.

The impression of a shape memory alone is therefore insufficient, and it must additionally be guaranteed that the “pre-programmed” end shape is achieved by means of an adjustment force that is suited to overcome the forces acting against the unfolding of the implant to obtain its end shape.

According to another aspect of the invention, the material suited for impressing the shape memory is a material with a shape memory that can be activated, preferably a material with an electrically, thermally, mechanically or magnetically activable shape memory.

This enables the cornea implant, once implanted, to be readjusted at any time, with the possibility that the activation may also lead to different end shapes.

When using materials with a thermally activable shape memory according to the invention, the cornea implant may also adopt its desirable end form after plastic deformation. In a preferable embodiment of the invention, this is achieved by the targeted use of shape memory alloys.

By using materials with an activatable shape memory as cornea implant according to the invention, it is moreover possible that the inserted cornea implant having reached its end shape may further change its end shape even after implantation, for example, by being activated through an electric or magnetic field created from outside or by having an electric current pass through it.

The group of materials with shape memory according to aspects of the invention also includes those which may change their size, or elasticity, or plasticity in the wake of swelling or deswelling, such as by absorption or extraction of water. Such materials may include completely or incompletely hydrated plastics, such as HEMA or hydrogels.

There are also materials with a shape memory where the end shape can be activated and adjusted by mechanical signals (e.g., by using ultrasound or reducing the frictional forces inside the pocket) or by chemical signals (e.g. change in pH value).

According to a further aspect of the invention, the materials with a shape memory are shape memory alloys, for example those based on Ni—Ti, Cu—Zn—Al and Cu—Al—Ni. According to an embodiment, such cornea implants are wrapped in an inert, biocompatible protective layer.

To enable a particularly exact automatic unfolding inside the cornea pocket, there is yet another embodiment of the invention, according to which the choice of a suited material and the respective adjustment to the geometrical form of the annular cornea implant assures that the deformability of the cornea implant is at least 25 percent in at least one outer dimension of the ring, preferably the ring diameter, and the cornea implant has an adjustment force (or restoring force) into the original ring form (or desired end shape) in the range of 0.001N to 1N, ideally between 0.01N and 0.5N, in at least one direction (the direction of deformation), but ideally in all directions.

A particularly preferable geometrical form of the annular cornea implant has proven to be a cornea implant shaped like a circular ring with an outer diameter ranging between 4 mm and 12 mm, a ring width between 0.4 mm and 1.5 mm, preferably 0.5 mm, and a ring height between 0.01 mm and 0.8 mm. To this end, the inner diameter of the ring should not lie within the pupil width to avoid the perception of disturbing effects at the border.

The front face of the cornea implant is preferably convex and the back face preferably concave. This assures that the pocket wall more or less flawlessly adheres to the cornea implant, with the front and back face of the ring seamlessly flowing into each other at the edges.

On account of the good deformability and sufficient adjustment force according to an aspect of the invention, the cornea implant may also have the shape of a closed or split ring and still be inserted into the cornea pocket via the narrow access without difficulty. The shape of a closed ring guarantees that the cornea implant, after being implanted in the cornea pocket, resumes an annular, undistorted and stable ring form.

The cornea implant may have the form of a circular ring as its end shape for the correction of shortsightedness, or a non-circular ring form for the correction of other refractive errors, such as astigmatism in the case of an elliptic ring form.

An object of a further aspect of the invention is to provide a corneal implant which is suited for introduction into the optical center of the human eye and which may be used to correct presbyopia on its own as well as presbyopia in combination with hypermetropia (farsightedness) or myopia (nearsightedness).

According to an aspect of the invention, this object is achieved through a corneal implant as disclosed herein. An aim is to provide the corneal implant with an effective thickness, measured in the direction of the optical axis of the eye, of more than 50 μm and a maximum width, measured in a plane perpendicular to the direction of thickness, of less than 1 mm, the corneal implant having no imaging function in relation to the human eye.

A corneal implant of the selected dimensions is on one hand suited for being positioned in the optical center of the human eye without impairing the vision of the human eye, and on the other also suited for correcting presbyopic vision by modifying the curvature of the cornea through corneal deflection in its optical center. Since a corneal implant according to an aspect of the invention has no imaging function in relation to the human eye, which means that it has no optical effect whatsoever, it is relatively easy to produce. The dimensions according to an aspect of the invention allow the introduction of the implant directly into the optical center of the eye without reducing its vision. Resulting from the central addition of volume an aspherical surface contour of the cornea may be produced in the surroundings of the corneal implant, which facilitates a multi-focal image so as to correct presbyopic vision. Corrections of hyperopic conditions are possible as well. The implantation in the optical center of the cornea implies that the implant, with due consideration of the finite defining accuracy of the optical center and the finite positioning accuracy of the implant in the cornea, is introduced into the cornea along a line that represents the optical center, i.e. the line along which the optical axis passes through the cornea.

Contrary to the state of the art, an implant according to this aspect of the invention deliberately fails to support optical imaging. The optical effect of the implant is thus indirectly achieved and determined by the contour of the transitional area in the adjoining tissue. Since the implant according to this aspect of the invention has no direct optical imaging function, it needs no optically designed surfaces. The implant surfaces may be flexibly shaped and are not bound by optical requirements such as in optically effective implants. The geometry of the implant is exclusively determined by geometrical considerations regarding the type of replacement of the tissue surrounding the implant. What was said also implies that an implant according to this aspect of the invention need not be transparent, but may also be opaque or partly transparent and of any sort of color in order to assume its function according to the invention. Since under certain conditions (geometry) disturbing surface phenomena may occur at the transparent faces (similar to optical implants), while the geometry may display a favorable tissue replacement behavior, this side effect may be eliminated by eliminating or reducing the transparency of the corneal implant. In case a color is to be added to the corneal implant, black has proven to be particularly advantageous as it does not stand out from the underlying black color of the pupil.

In any case, in an implant according to an aspect of the invention, even if it were made of transparent material, the proportion of light rays entering the implant after its introduction into the cornea in no way contributes to the perception of a retinal image, which means it fails to project a perceivable image on the retina. Among other things, this stems from the dimensions and measurements of the implants according to aspects of the invention, their geometry, their surface properties, their material, their color, optical losses occurring along the area of contact with the surrounding tissue as well as biological interaction with the surrounding tissue. This is particularly the case if the implant has been embedded in the tissue for a certain period of time. This, in particular, makes the effect of the implant insensitive to optical and biological surface phenomena, by which it differs from state-of-the-art products.

Since the eye complies with the laws of geometrical optics, and the latter basically corresponds to the optics of the rays close to the axis, an expert would expect that by introducing a non-optical body—typically representing an optical obstacle—into the optical center of the cornea, the vision would be substantially impaired. Surprisingly, it could be shown that if this body has the characteristics according to the invention, such impairment will only be minimal.

In a further aspect of the invention, the ratio between width and effective thickness of an implant is less than three and/or more than one. It has been shown that in this case particularly positive results regarding multi-focality can be achieved.

Effective thicknesses of less than 500 microns and width variations not exceeding 30 percent of the largest width also help to adjust the outline of the corneal surface to the requirements of multi-focal imaging.

In another aspect of the invention, the corneal implant is rotation-symmetrically arranged around the axis along the effective thickness. In one embodiment, the corneal implants have the shape of a sphere, thus assuring an ideal formation of the aspherical surface contour on the corneal surface.

It is a further object of an aspect of the invention to provide a method for the correction of impaired vision in the human eye, in particular for the correction of presbyopia on its own or in combination with hypermetropia, by inserting a corneal implant into the optical center of the human eye without risking that the function of the implant is impaired by deposits in the optical center of the cornea around the implant and without the need to use sophisticated optical lenses as implants.

An aim is to introduce into the optical center of the cornea of the human eye one or several corneal implants without an imaging function in relation to the human eye, each of which has an effective thickness of more than 50 microns, measured in the direction of the optical axis of the eye, and a maximum width of less than 1 mm, measured in a plane perpendicular to the direction of thickness, with the purpose of modifying the curvature of the corneal surface around the optical center of the cornea through deflection of the corneal surface in the optical center.

Another object of an aspect of the invention is to provide a method for the correction of impaired vision in the human eye, in particular for the correction of presbyopia in combination with myopia, by introducing a corneal implant into the optical center of the human eye without risking that the function of the implant is impaired by deposits in the optical center of the cornea around the implant and without the need to use sophisticated optical lenses as implants.

According to an aspect of the invention, this task is achieved through a method disclosed herein. An aim is to introduce into the optical center of the cornea one or several corneal implants without an imaging function in relation to the human eye, which have an effective thickness of more than 50 μm, measured in the direction of the optical axis of the eye, and a maximum width of less than 1 mm, measured in a plane perpendicular to the direction of thickness, for the purpose of accomplishing a deflection of the surface of the cornea in its optical center; several corneal implants, preferably one ring-shaped corneal implant, are additionally positioned outside the optical center of the cornea, assuring that the curvature of the cornea outside the optical center is modified.

As further aspect of the invention relates to methods for treating corneal disease. In particular, aspects of the invention relate to methods for treating corneal disease, such as keratoconus, by forming a corneal pocket in the cornea, introducing a corneal stiffening substance into the corneal pocket and irradiating the cornea with electromagnetic radiation.

In one aspect of the invention, a method for treating corneal disease includes the steps of forming a corneal pocket in a cornea at a depth from a corneal surface, introducing a corneal stiffening substance into the corneal pocket and irradiating the cornea with electromagnetic radiation.

In further aspects of the invention, the corneal pocket may be formed at a depth of between approximately fifty microns and four hundred fifty microns from the corneal surface, preferably between approximately two hundred fifty microns and three hundred fifty microns from the corneal surface, and more preferably approximately three hundred microns from the corneal surface.

In a further aspect of the invention, the corneal pocket may have a diameter of between approximately two millimeters and ten millimeters.

In a further aspect of the invention, the step of forming a corneal pocket may includes forming a tunnel-like entry through the corneal surface. The tunnel-like entry may have a width of less than approximately six millimeters and preferably less than approximately five millimeters. The corneal stiffening substance may be introduced into the corneal pocket thorough the tunnel-like entry.

In further aspects of the invention, the corneal pocket is formed using a manual dissector, a mechanical microkeratome or a laser.

In a further aspect of the invention, the cornea may be irradiated with ultraviolet light, preferably ultraviolet A light.

In a further aspect of the invention, the cornea may be irradiated with electromagnetic radiation for less than thirty minutes. The cornea may be irradiated with electromagnetic radiation without removing the epithelium of the cornea.

In a further aspect of the invention, the method for treating corneal disease may additionally include the step of inserting a corneal implant into the corneal pocket. The corneal implant may be inserted into the corneal pocket thorough the tunnel-like entry. The corneal implant may be a continuous ring implant, a split ring implant and/or a compressible implant.

In a further aspect of the invention, the corneal implant may be inserted into the corneal pocket before the corneal stiffening substance is introduced into the corneal pocket and before the cornea is irradiated with electromagnetic radiation.

In a further aspect of the invention, the corneal implant may be inserted into the corneal pocket after the corneal stiffening substance is introduced into the corneal pocket and before the cornea is irradiated with electromagnetic radiation.

In a further aspect of the invention, the corneal implant may be inserted into the corneal pocket after the corneal stiffening substance is introduced into the corneal pocket and after the cornea is irradiated with electromagnetic radiation.

An advantage of a method for treating corneal disease according to an aspect of the invention is that the epithelium of the cornea is preserved. A further advantage of a method for treating corneal disease according to an aspect of the invention is that a painless method for stopping the progression of corneal disease is provided. A further advantage of a method for treating corneal disease according to an aspect of the invention is that the method provides the option of effectively correcting even higher grades of corneal irregularities than can be corrected using prior known methods.

A further aspect of the invention relates to a method for treating refractive errors and vision disorders of an eye. In particular, aspects of the invention relate to methods for treating refractive errors and vision disorders of the eye including the steps of fixing a suction ring to an eye, applanating a cornea of the eye, guiding a blade into the cornea with a guiding mechanism, creating a pocket inside the cornea by guiding the blade inside the cornea, removing the blade from the cornea and removing the suction ring from the eye.

In a further aspect of the invention, the method for treating refractive errors and vision disorders of the eye may include the step of providing the blade with a pointed tip.

In a further aspect of the invention, the method for treating refractive errors and vision disorders of the eye may include the step of connecting the suction ring and the blade with the guiding mechanism.

In a further aspect of the invention the blade may comprise a non-metallic blade.

In a further aspect of the invention, the method for treating refractive errors and vision disorders of the eye may include the step of vibrating the blade as the blade is guided into the cornea with the guiding mechanism and/or vibrating the blade as the blade is guided inside the cornea to create the pocket inside the cornea.

In a further aspect of the invention, the blade does not touch the suction ring or the guiding mechanism as the cornea is being cut.

In a further aspect of the invention, the method for treating refractive errors and vision disorders of the eye may include the step of inserting an implant or inlay into the pocket inside the cornea. The implant or inlay may have a ring shape, such as a continuous ring shape or a non-continuous ring shape. The ring-shaped implant or inlay may be compressible and may have a shape memory.

In a further aspect of the invention, the implant or inlay may have no dimensions larger than 1 millimeter and/or may have no direct optical function.

In a further aspect of the invention, the implant or inlay may be positioned inside the pocket inside the cornea. The implant or inlay may be positioned in a center of the cornea and/or a center of a visual axis.

In a further aspect of the invention, the method for treating refractive errors and vision disorders of the eye may include the step of introducing a corneal stiffening substance into the pocket inside the cornea.

In a further aspect of the invention, the method for treating refractive errors and vision disorders of the eye may include the step of irradiating the cornea with electromagnetic radiation.

### BRIEF DESCRIPTION OF THE DRAWINGS

Other benefits and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a side view in partial section of an exemplary device according to an aspect of the invention, which device may be used for forming a corneal pocket in a cornea in a method according to an aspect of the invention;

FIG. 2 shows a side view in partial section of a second exemplary device according to an aspect of the invention, which device may be used for forming a corneal pocket in a cornea in a method according to an aspect of the invention;

FIGS. 3a through 3c show a top view of the direction of the cuts drawn by a blade in forming a corneal pocket in a method according to an aspect of the invention, such as by a device as shown in FIG. 1 or 2;

FIG. 3d shows a top view of a cornea with a corneal pocket produced by the cuts illustrated in FIGS. 3a through 3c;

FIG. 4a shows a cross section of a cornea flattened by applanation with a blade being introduced for producing a pocket;

FIG. 4b shows the pocket of an unimpinged cornea produced according to FIG. 4a in cross section;

FIG. 5 shows the attachment of the blade to the holding device comprising a forked blade guide;

FIGS. 6a through 6c show incisions made to cut out a lens-shaped portion of tissue by using applanators with differently curved contact faces in cross section;

FIG. 7 shows a construction variant of an applanator with a curvable contact face in an enlarged scale;

FIGS. 8a through 8g show side views of different applanators for devices and methods according to aspects of the invention;

FIG. 9 shows a top and a side view of a blade for devices and methods according to aspects of the invention;

FIG. 10 shows a top view of a device into which a transparent applanator has been inserted;

FIG. 11 shows a sectional view of a cornea implant according to an aspect of the invention;

FIG. 12 shows a cornea implant according to an aspect of the invention which has been deformed for the purpose of inserting it via a tunnel-shaped access;

FIG. 13 shows a cornea implant according to an aspect of the invention with different annular cross sections along the circumference;

FIG. 14 shows a cornea implant according to an aspect of the invention with a saddle-shaped ring geometry;

FIGS. 15a and 15b shows two cornea implants according to aspects of the invention with different annular shapes;

FIGS. 16a through 16j show different cross sections of cornea implants according to aspects of the invention;

FIGS. 17a thorough 17d show a cornea implant according to an aspect of the invention having the shape of a closed or continuous circular ring;

FIGS. 18a through 18d show a cornea implant according to an aspect of the invention having the shape of a split or non-continuous circular ring;

FIGS. 19a and 19b show an annular cornea implant with a central lens body according to an aspect of the invention;

FIG. 20 is an illustration of an implantation process wherein an annular cornea implant according to an aspect of the invention is inserted into a cornea pocket via a narrow tunnel;

FIG. 21 shows cross section of the cornea of a human eye with an implanted corneal implant according to an aspect of the invention;

FIG. 22 shows a cross section of the cornea of a human eye with an implanted corneal implant according to an aspect of the invention, wherein the different effective areas are marked;

FIG. 23 shows a cross section of a corneal implant according to an aspect of the invention;

FIG. 24 shows a cross section of an alternative corneal implant according to another aspect of the invention;

FIGS. 25a through 25s show further alternative cross sections of corneal implants according to aspects of the invention;

FIGS. 26a and 26b show a corneal implant according to an aspect of the invention in combination with a ring-shaped corneal implant;

FIGS. 27a and 27b show a corneal implant according to an aspect of the invention in combination with several individual corneal implants;

FIGS. 28a and 28b show a corneal implant according to an aspect of the invention in combination with several individual aligned corneal implants;

FIG. 29 shows a cross section of a cornea with a corneal pocket formed therein for introduction of a corneal stiffening substance in a method according to an aspect of the invention; and

FIG. 30 shows a top view of the cornea illustrated in FIG. 29;

### DETAILED DESCRIPTION OF THE DRAWINGS

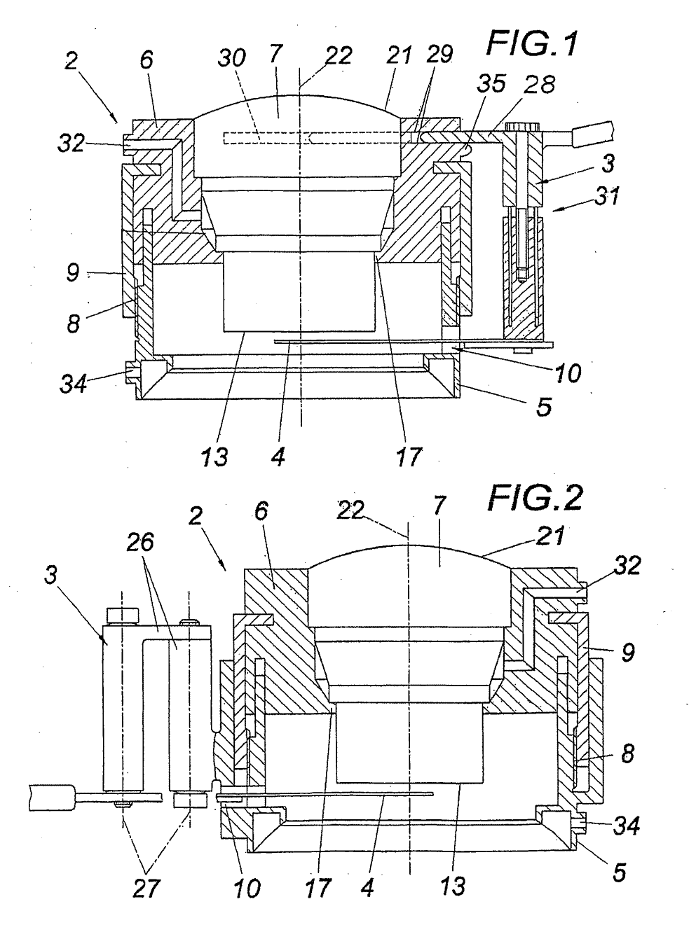

Pursuant to the exemplary embodiment in FIG. 1 and the exemplary embodiment in FIG. 2, the devices for cutting a cornea 1 of an eye to correct its refractive power according to the invention generally comprise a frame 2 and a holding device 3 for supporting a blade 4. The frame 2 has a fixation ring or suction ring 5, which may be drawn onto the eye, and a receptacle 6, which may be coaxially displaced relative to the fixation ring or suction ring 5 and which serves to accommodate an applanator 7 for deforming the cornea within the fixation ring or suction ring 5. The cornea 1 thus projects through the fixation ring or suction ring 5, within which, in particular offset in height relative to the fixation ring or suction ring 5, the applanator 7 for impingement of the cornea is located. The fixation ring or suction ring 5 is furnished with a thread 8 for coaxial displacement, in which a nut 9 engages that is mounted on the receptacle 6 allowing rotation. The radial displacement of the applanator could also be performed by displacing only the applanator within the receptacle. For example, the coaxial displacement can be achieved also only by the coaxial displacement of the applanator within the frame (receptacle) when the receptacle 6 is fixed relative to the suction ring 5 or by the use of different frames or by the use of different applanators.

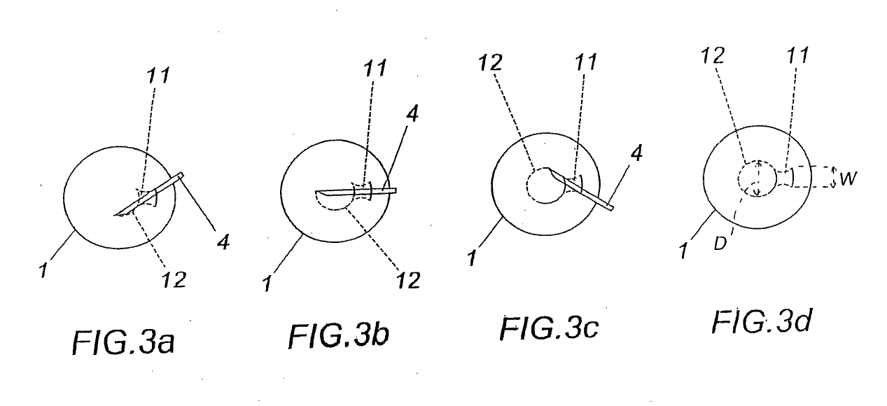

By rotating the nut 9, the receptacle 6 and/or the applanator 7 may thus be displaced relative to the fixation ring or suction ring 5 and/or the cornea 1. The holding device 3 for supporting the blade 4 is guided on the frame 2 in a plane that is perpendicular to the axis of the fixation ring or suction ring 5, and the blade 4 passes through the frame 2 via a peripheral recess 10 with clearance and is mounted in front of the applanator 7. The blade 4 is, in particular, guided by the holding device 3 in such a way that said blade 4 is radially displaceable relative to the fixation ring or suction ring 5 as well as movable around an axis perpendicular to the guiding plane via the holding device 3, for the purpose of cutting a pocket 12 through a merely tunnel-like entry 11 into the corneal tissue, as may in particular be inferred from FIGS. 3a through 3c.

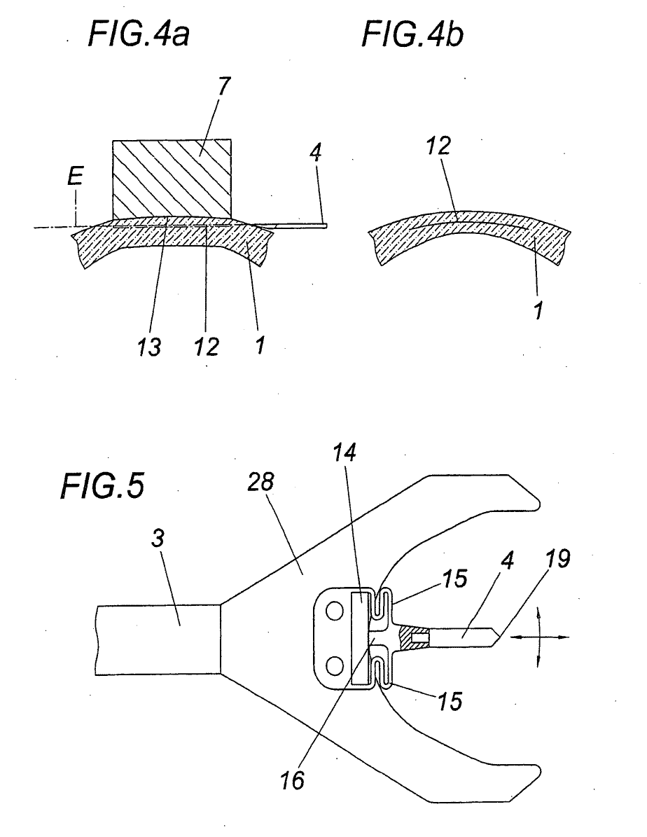

It is also conceivable that the cutting plane E of the blade 4 also lies in the guiding plane of the blade 4. If a pocket 12 is now to be cut into the cornea 1 (FIG. 4a), an applanator 7 is first pressed onto the corneal surface in such a way that the cornea 1 is deformed in a defined manner corresponding to the contact face 13 of the applanator 7, whereby the shape of the applanator 7 is embossed on the cornea 1. Through said impingement of the cornea 1 a correspondingly large pocket 12 may then be cut into the cornea 1. Once the applanator 7 has been placed on the cornea 1 accordingly, the tip of the blade 4 is placed on cornea 1 and the outer tissue layers of the cornea 1 are penetrated in order to produce a tunnel-like entry 11. It is of decisive importance in this case that the blade 4 does not slip from where it is placed on the corneal surface. The invention therefore in one aspect comprises a blade 4, which passes through the frame recess 10 with clearance, and the holding device 3 supports a vibrator 14 for setting the blade 4 in oscillatory motion in the cutting plane E, as may be seen in FIG. 5. By passing through the frame recess 10 with clearance, the blade 4 does not rest on the frame as in the other state-of-the-art techniques, so that damage to the blade 4, particularly to the edge of the blade 4, may be precluded.

Thus, even extremely hard and brittle materials may be used for the blade 4, and because of the resulting sharpness of the blade 4 slipping of the latter as it penetrates into the cornea 1 may virtually be ruled out. However, it has been shown that notwithstanding said blades 4, great force is required to penetrate the outer layer of the cornea 1, which according to an aspect of the invention is facilitated by an oscillatory motion of the blade 4. For this purpose, the holding device 3 is preferably furnished with a vibrator 14 designed as a piezo element, which vibrates in the cutting plane E of the blade 4. Said vibrator 14 impinges upon the blade 4, which is attached to the holding device 3 by means of springs 15, with the aid of a web 16 pressing against the piezo element. However, using an unbalanced motor instead of the piezo element is also conceivable.

Once the tunnel-like entry 11 has been cut through the outer tissue layers of the cornea 1, the blade is guided for the purpose of cutting a pocket 12 in such a way that any further contact with the corneal surface is avoided. Therefore, the pocket 12 produced via the tunnel-like entry 11 is merely cut inside the cornea 1, and it goes without saying that the vibrator 14 further facilitates the cutting process and also allows a highly accurate way of cutting. The pocket 12 and the tunnel-like entry 11 may be adequately widened by using a suitable instrument, whereby the introduction of implants may be facilitated. Such implants may include inlays or any other devices intended to be implanted in or on the cornea.

With the aid of yet another device, an implant which is preferably foldable or deformable may be introduced into the implant bed in the cornea 1 through the tunnel-like orifice 11. The implant then unfolds in the pocket 12 to take on the desired shape.

Since according to an aspect of the invention a highly exact pocket 12 may be produced inside a cornea 1, it is possible, among other things, to implant lenses which are standardized in terms of their radius of curvature in the area of the optical center. This is of significance as the natural curvature of the cornea varies between approximately 7 mm and 9 mm and through a flat, uncurved impingement of the cornea 1, a defined radius of curvature of the pocket 12 may not be achieved. By using an applanator 7 with a specifically curved contact face 13 and determining a specific cutting depth, the radius of curvature of the pocket 12 to be produced may be accurately defined. This allows lenses that are standardized in terms of the curvature of their base to be implanted in all patients, which results in significant cost savings in lens manufacturing.

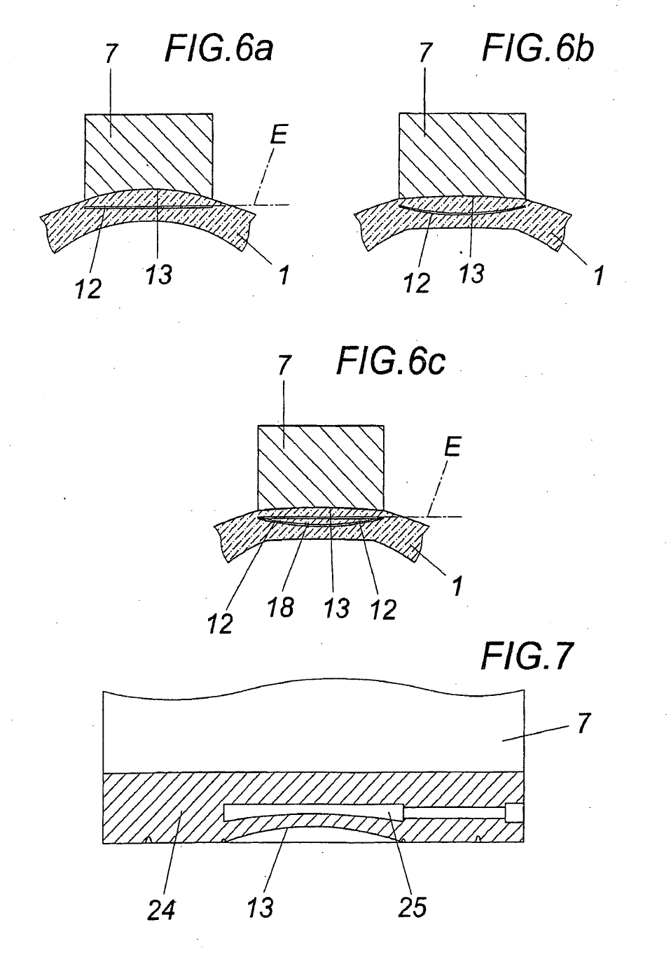

The receptacle 6 accommodating the applanator 7 is designed as a receptacle for the stop-delimited receipt of interchangeable applanators 7 with differently curved contact faces 13 for applanation of the cornea. A stop 17 is in particular illustrated in FIGS. 1 and 2. This makes it easy to produce pockets 12 for delimiting a lens-shaped portion of tissue 18 by performing sequential incisions. Especially given the fact that the frame 2 need not be withdrawn from the eye while exchanging applanators, the use of a receptacle for interchangeable applanators ensures that a lens-shaped portion of tissue 18 with a predefined size is always cut out, which is not the case if the conventional devices are used. The lens shaped portion of tissue 18 may be produced according to FIGS. 6a through 6c. After cutting an initial pocket into the corneal tissue, as is also described above, the blade 4 is removed from the corneal tissue (FIG. 6a) and a second pocket 12, embossed by another applanator 7 having a differently curved contact face 13, is incised (FIGS. 6b and c). For this purpose, the applanator 7 either needs to be exchanged or altered with respect to its contact face 13, as may, for example, be achieved using an applanator 7 according to FIG. 7.

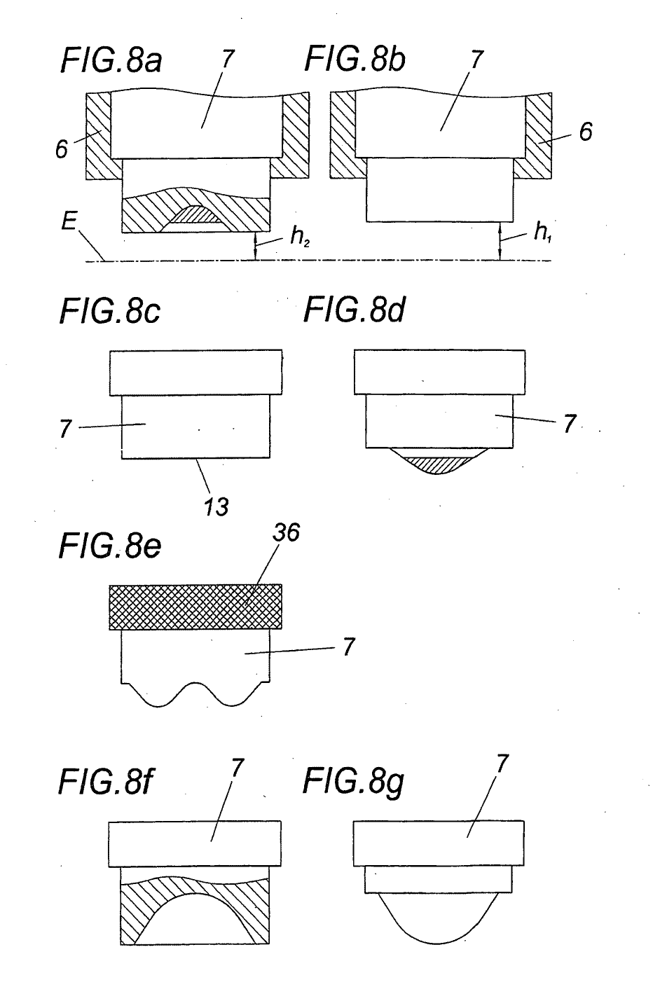

By performing a new cut through the tunnel-like entry 11, apart from a new pocket 12 also a lens-shaped portion of tissue 18 (FIG. 6c) is incised, which may be pulled out through the tunnel-like entry 11, for example, by using tweezers. The lens-shaped portion of tissue in its shape corresponds to the difference of the dissimilarly curved contact faces 13 of the two applanators 7 and/or the desired dioptric change through a corresponding alteration of the anterior corneal curvature. The applanator 7 additionally has a grip 36 with an engraved surface in its upper area (FIG. 8e), which makes the applanator 7 easily replaceable. It is useful in this case to ensure that the applanator 7 projects out of the receptacle 6 in order to be grasped easily.

To facilitate the cutout of a lens-shaped portion of tissue 18, the second cut of the blade 4 may be performed somewhat deeper into the cornea 1 (FIGS. 8a and 8b). This may be achieved by ensuring that the perpendicular distances h1 and h2 of the peripheral edges of the contact faces 13 of the applanators 7, which are sequentially inserted in the receptacle 6, are different with respect to the cutting plane E of the blade 4. Exemplary embodiments of different contact faces 13 of applanators 7 are shown in FIGS. 8a through g. The dimensions of the lens-shaped portion of tissue 18 that is cut out using the applanators shown in FIGS. 8a and 8b as well as 8c and 8d are indicated by crosshatching.

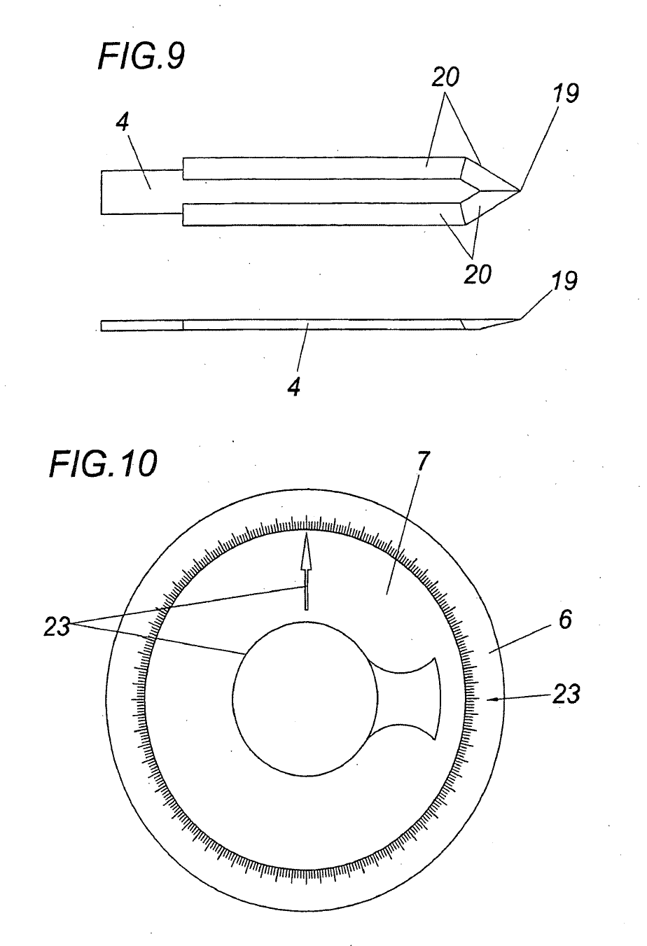

The preferably non-metallic blade 4 has a pointed tip 19 with two edges 20 originating from this tip as shown in FIG. 9. This specific type of tip 19 has proven useful for penetrating the outer layer of the cornea 1 and/or producing a tunnel-like entry 11. The shape of the blade 4 resembles that of a double-edged knife. Particularly advantageous cutting properties are obtained if the blade is made of diamond material, having a maximum width of 2 mm, a maximum thickness of 200 microns and a blade length of at least 8 mm, preferably 10 mm.

The applanators 7 are preferably made of transparent material, such as plastic or glass, and are designed as enlargement lenses 21 as illustrated in FIGS. 1 and 2, with their focal point lying in the area of the contact face 13, preferably on the axis of symmetry of the applanator 22. An applanator 7 being designed in such a way makes it relatively easy for a surgeon to monitor the progress of treatment of the cornea 1.

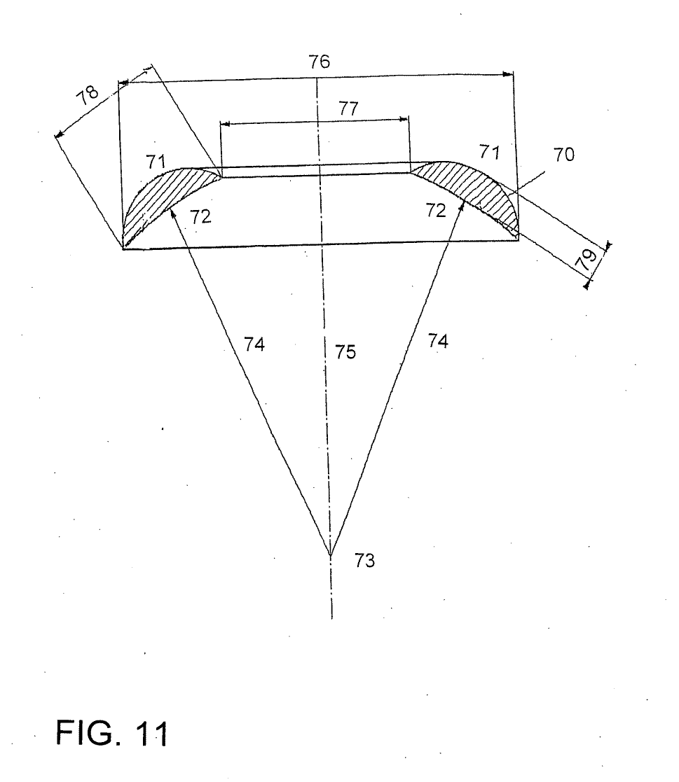

The transparent applanator 7 has markings 23 on its side facing the eye. These markings 23 enable the surgeon, for example, to optimally orientate himself as to where to introduce the blade 4 for cutting a tunnel-like entry. Markings 23 may also be applied for the optical treatment zone to indicate to the surgeon the boundaries of the pocket 12 to be cut. The transparent applanator 7 also has markings 23 on its side diametrically opposite the eye, which are related to markings 23 on the receptacle 6 for accommodating the applanator 7 (FIG. 10). This enables the surgeon, by offsetting the applanator 7 in relation to the receptacle 6, to perform refractive power corrections, particularly those related to astigmatism.

The contact face 13 of the applanator illustrated in FIG. 7 is made of a deformable material 24, which may be curved via an actuator 25 to obtain different shapes that are maintained. Through a supply line linked to the actuator 25, compressed gas and compressed fluid may be fed into the cavity of the actuator 25, whereby the contact face 13 of the applanator 7 may be curved so as to obtain different shapes that are maintained.

According to the exemplary embodiment illustrated in FIG. 2, the holding device 3 consists of a lever system comprising at least two lever arms 26 having pivot axes 27 which are perpendicular to the cutting plane E of the blade 4; one lever arm 26 receives the blade 4 and the other lever arm 26 is linked to the frame 2, preferably to the receptacle 6.

According to the exemplary embodiment illustrated in FIG. 1, the holding device 3 may also comprise a forked blade guide 28 receiving the blade 4, which is guided, possibly without clearance, between parallel faces 29 of a peripheral groove 30 provided on the frame 2, in particular on the receptacle 6. The blade 4 is offset to the forked blade guide 28, with the distance between the cutting plane E of the blade 4 and the contact face 13 of the applanator 7 being adjustable by means of a position adjuster 31 having the shape of a winding gear. A knob 35 is provided on the receptacle 6 to allow the forked blade, guide 28 to be easily pushed in.

The applanator 7 may be fixed into the receptacle 6 by means of a partial vacuum. For this purpose, air may be sucked out of a chamber that is located between the receptacle and the applanator through a line 32. The applanator has the shape of a truncated cone, which allows easy insertion of the applanator. Furthermore, it is conceivable that other mechanical holding devices instead of the pressure line are used, including bayonet closures, magnetic, electromagnetic, hydraulic, or other equivalent mechanisms. A similar procedure is conducted when drawing the fixation ring or suction ring 5 onto the eye by a pressure line 34.

As seen in FIG. 11, a cornea implant according to an aspect of the invention comprises an annular ring body 70, which may have a convex front face 71 and a concave back face 72. The concavity of the back face 72 may be spherical or aspherical, such as by approximation via several spherical curvatures. The centers of curvature 73 of the radii of curvature 74 of the two back face sections 72, 72 are preferably arranged alongside the axis 75 of the cornea implant according to an aspect of the invention. In aspherical back faces, several centers of curvature 73 are arranged alongside the axis 75, which corresponds to the optical axis.

Apart from allowing a perfect adjustment of the back face of the ring to the implantation bed and preventing the formation of deposits and pressure atrophies in the tissue, this also drastically increases the deformability of the rings, without reducing the adjustment force in the same way.

The outer diameter 6 of the cornea implant is preferably in the range of 4 mm to 12 mm, ideally between 5 mm and 9 mm. The inner diameter 77 is preferably in the range of 3 mm to 11 mm, ideally between 4 mm and 8 mm. The ring width 78 is ideally 0.5 mm, ought to be less than 1 mm to ensure a proper nutrient supply, and is preferably between 0.4 mm and 1.5 mm.

The ring height 79 is in the range of 0.01 mm to 0.8 mm, ideally between 0.1 mm and 0.4 mm.

The inner diameter 77 of the cornea implant should in any case be wider than the respective pupil width to avoid effects at the border which the patient may find disturbing.

The front and back faces 71 and 72 flow seamlessly into each other at the edges.

The back face 72 ideally follows the natural course of the corresponding pocket wall inside the cornea (local corneal radii minus pocket depth plus (or by) correction factor, which considers the deformation of the cornea resulting from the insertion into the pocket), so that the concave progression of the back face of the ring corresponds to a spherical or aspherical curvature, with radii 10 lying between 4 mm and 40 mm, preferably between 6 mm and 10 mm.

According to an aspect of the invention, the cornea implant 70 has a shape memory which is impressed on the basis of its material and/or geometry and designed in such a way that the cornea implant has a deformability from a starting shape, which enables the insertion of the cornea implant into the cornea pocket via the narrow, preferably tunnel-shaped access 11 with an inner width of less than 5 mm and concurrently has an adjustment force in the end shape thereof, which enables an essentially independent unfolding of the cornea implant in the cornea pocket 12.

The preferred materials for this purpose may include plastics such as PMMA, HEMA, silicone, polycarbonates, polyethylene or other polymer plastics or shape memory alloys.

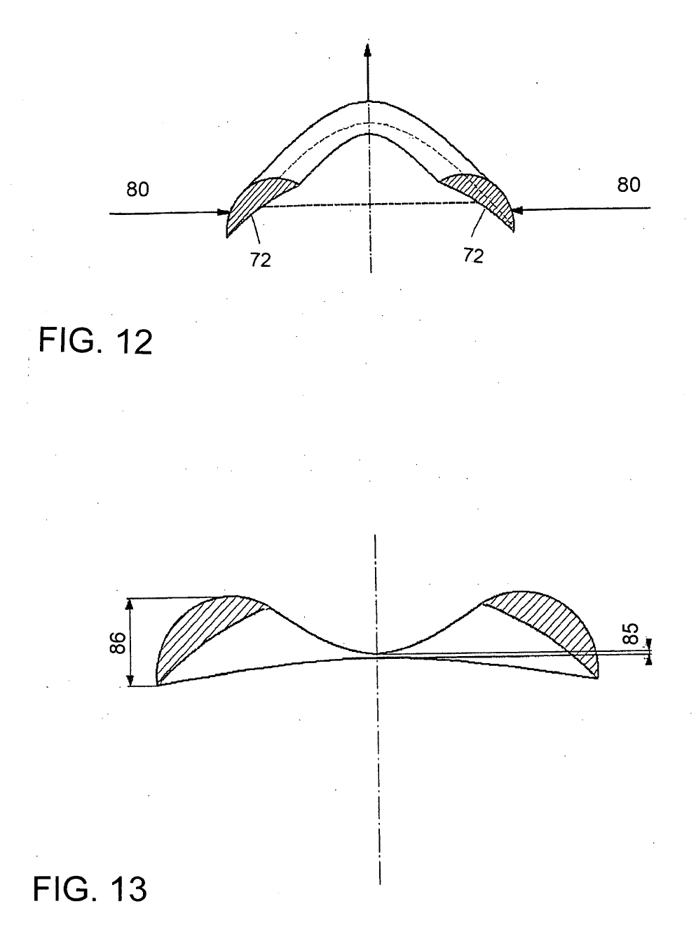

FIG. 12 shows a cornea implant according to an aspect of the invention, which is laterally compressed by force, as symbolized by the arrows 80, to be able to be inserted via the narrow access (not included) into a cornea pocket.

With respect to the elastic deformation of materials, the adjustment force is in general reciprocally proportionate to the deformability.

When using materials which are only elastically deformable within a very narrow deformation range (e.g. PMMA), it is essential to give the cornea implant a special shape by which it becomes sufficiently deformable yet also retains its restoring force.

In the case of a cornea implant as illustrated in FIG. 12, this is achieved by the special contour line of the back face 72 towards the ring center, where the centers of curvature 73 of the radii 74 are arranged along the axis 75, thus not only achieving a simple oval shape, but a saddle-shaped object as a part of the ring deviates from the ring level into the third dimension 81 when being deformed (compression). This generates an additional degree of freedom, which considerably increases the deformability without essentially reducing the adjustment force. This is in the case of a continuous, split or incomplete ring implant. In particular also in the case of a split ring implant when the touching ends do not touch anymore in the compressed state. In a particular embodiment, a part of a split ring implant overlaps another part at a different level with respect to the implant plane (plane of the implant in an uncompressed state) (3-dimensional). The split contour of the split ring can be any shape but is preferably a virtually linear splitting line with an angulation of 30° with respect to the implant plane (ideally between 10° and 60°, for example, 20°, 25°, 40°, 45° or 50°).

According to an aspect of the invention, the deformability must be at least 25 percent of a typical annular dimension and the cornea implant has an adjustment force into the original ring form in the range of 0.001N to 1N, ideally between 0.01N and 0.5N.

A typical ring dimension in a circular cornea implant, for instance, is the ring diameter 90, whereas in an elliptic cornea implant, for instance, it is the minor axis 89 (FIGS. 5a and b).

In the embodiments according to FIG. 11 and FIG. 12 it is even possible to deform a ring made of PMMA (a material that is difficult to deform and easily breaks when being deformed), which has an outer diameter 6 of 5 mm, a ring width 8 of 0.5 mm and a ring height 8 of 0.25 mm as well as a radius of curvature 10 of 8 mm, by approximately 50% of its diameter, yet assuring that it does not break and retains a sufficient adjustment force to freely and independently resume its original ring geometry after being implanted in the cornea pocket against the forces resisting its unfolding. In this case, the impression of the shape memory results exclusively from a special geometry, whilst the choice of material is of no relevance.

To enable an exact fitting of the adjustment force, the cornea implant may have different ring cross sections 85, 86 along the circumference, as is illustrated in FIG. 13.

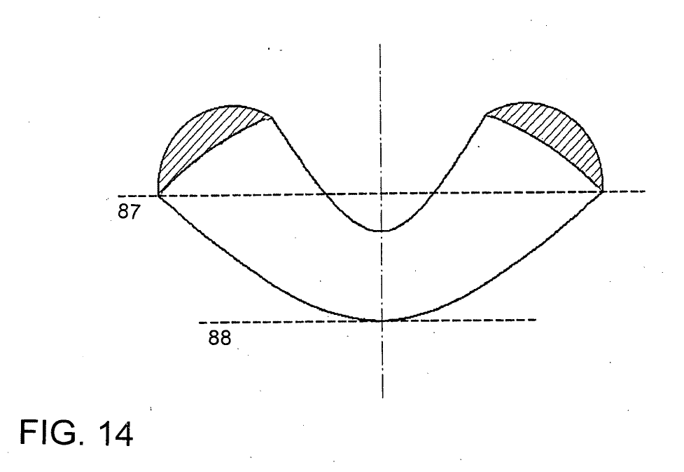

When it has the form of a “saddle”, the basic edges 87, 88 along the ring circumference are not on the same level, as can be seen in FIG. 14.

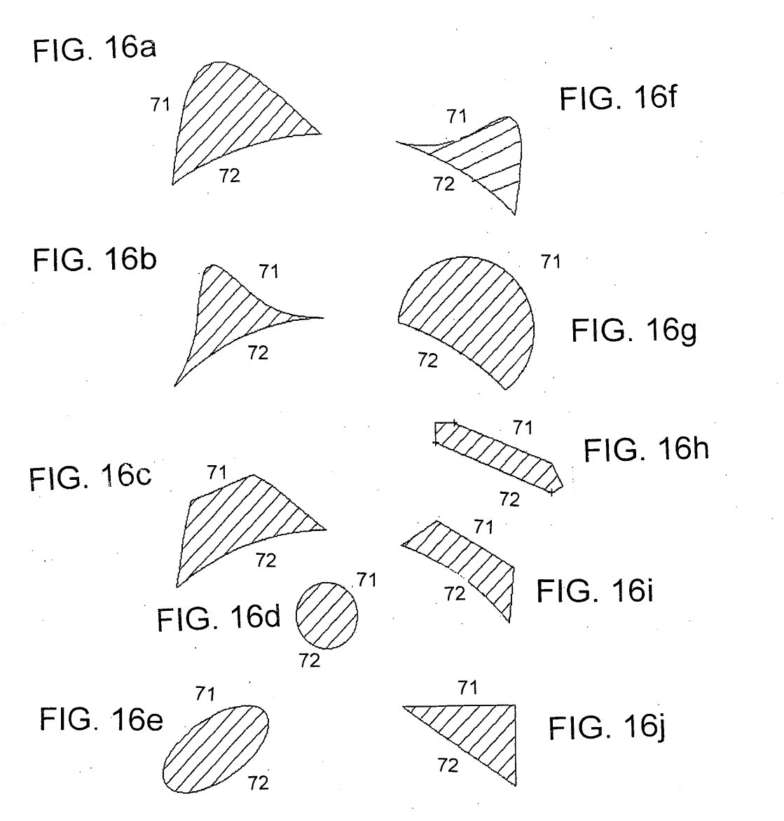

Depending on the refractive error to be corrected, a cornea implant 70 according to an aspect of the invention may have all sorts of different cross sections, as can be seen in FIGS. 16a-16j.

An exact attuning of the material and geometry of the cornea implant 70 to each other assures that the cornea implant may unfold independently inside the cornea pocket after insertion.

The respective material may be a material with a shape memory that can be activated, preferably a material that can be electrically, thermally, mechanically or magnetically activated, such as shape memory alloys.

The trigger signal for such activation may for instance be the temperature, so that the cornea implant according to the invention is sufficiently deformable in a cooled-down condition for inserting it into the cornea pocket via the narrow, preferably tunnel-shaped access. The cornea implant warms up inside the cornea pocket until reaching a certain trigger temperature, where the cornea implant resumes its end shape or, being exposed to an adequate adjustment force between 0.001N and 1N, ideally between 0.01N and 0.5N, approaches its end shape. Since the temperature inside the cornea may not be sufficient to achieve the trigger temperature, heat may also be introduced from outside the eye.

In the case of a material with a shape memory that can be magnetically or electrically activated, the material may, for instance, contain elements based on, or entirely consist of, Ni—Mn—Ga. After the successful implantation in the cornea pocket according to the invention, an adjustment from one end shape into another end shape may be achieved by inducing a voltage to the implant or exposing it to an electric or magnetic field. This even enables a posterior fine-tuning or adjustment of dioptres, such as would be necessary if the dioptres of the eye were to change again over time, without the need to replace the cornea implant.

In principle, different trigger signals may be used to activate the shape memory, including mechanical or chemical trigger signals. So when using certain materials, such as those appropriate due to their elastic deformability, the assumption of a defined end shape can be fostered by applying ultrasound or exerting pressure on the cornea implant or the bed of the implant. Moreover, by changing the degree of swelling of the implant, such as by adding or extracting liquid, the assumption of a shape may be facilitated or a respective activation energy required for obtaining a certain shape may be overcome. Moreover, such a trigger signal may also be released by an intentional pH value change in the tissue surrounding the implant or in the cornea implant proper.

Typical materials with a shape memory that can be activated are for instance polymer metals, ionic polymer-metal composites, IMPC, electroactive polymers (e.g. electronic or ionic EAP materials) such as polyacrylonitril (PAN), ceramics or electroactive ceramics, suited conductor or semi-conductor plastics, ionic polymeric conductor composites, IPCC, and magnetic shape memory elements such as those based on NiMnGa or Ni2MnGa.

Shape memory alloys may be alloys based on Ni—Ti, Cu—Zn—Al, Cu—Al—Ni and other materials. Plastics with a temperature-dependent shape memory as well as shape memory alloys may be easily deformed (in particular also by plastic deformation) preferably below a specific transformation temperature, but resume a predefined shape above this transition temperature. In this manner, changing the temperature of the folded cornea implant in its intermediate shape may help to achieve its unfolding into the desired shape after being inserted into the stromal implantation bed.

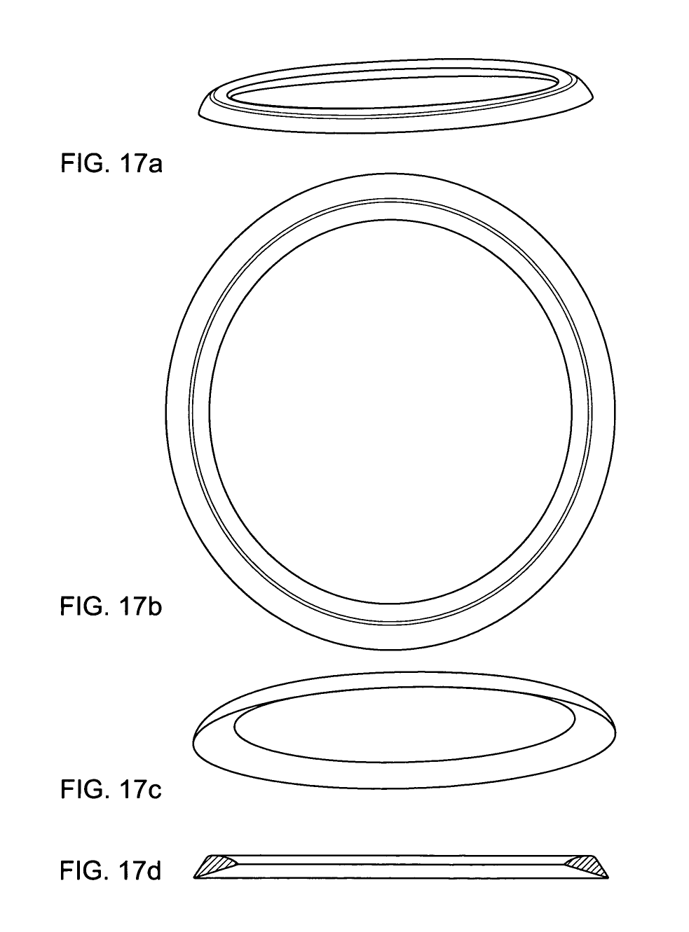

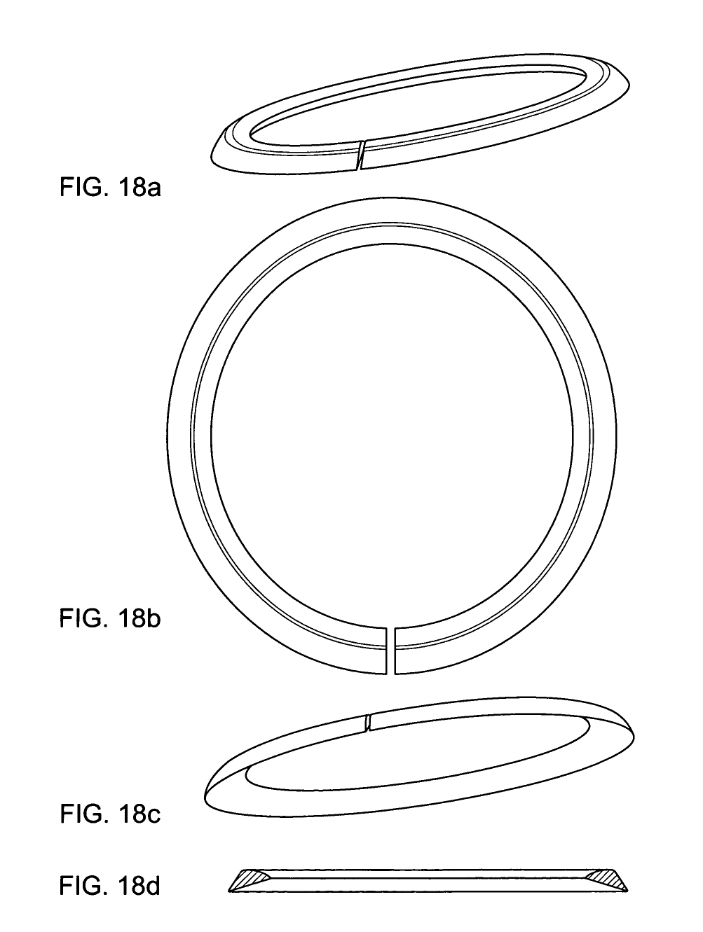

Shape memory alloys are characterized by a martensitic phase transition (second-order phase transition), where the material changes its crystalline structure when a specific transformation temperature is exceeded and assumes a predefined shape. This is an atomic and not a molecular phenomenon, such as in elastic deformation, where mostly polymer molecules are deformed. When a transformation temperature is exceeded, in fact, the atoms of the material spontaneously adopt an entirely different order. Although some shape memory alloys are biocompatible, shape memory alloys may be coated with silicone or any other inert, biocompatible materials (e.g. plastics) to assure that the tissue is not directly exposed to the alloy and thus cannot suffer any damage. Such cornea implants are also able to create pressure inside the tissue, such as when pressure is exerted against the cutting edges, which may additionally result in any kind of deformation of the corneal surface. Such cornea implants may have any shape, in particular they may be open or closed, annular (see FIGS. 17a-17d and 18a-18d), elliptic, saddle-shaped with one or more saddles, spiral-shaped, single- or multi-threaded, curved or straight, etc. The implants may or may not have a plastic coating. They may be continuous or segmented. They may be furnished with electrical contact elements. The majority of such elements are also suited for implantation into circular tunnels.

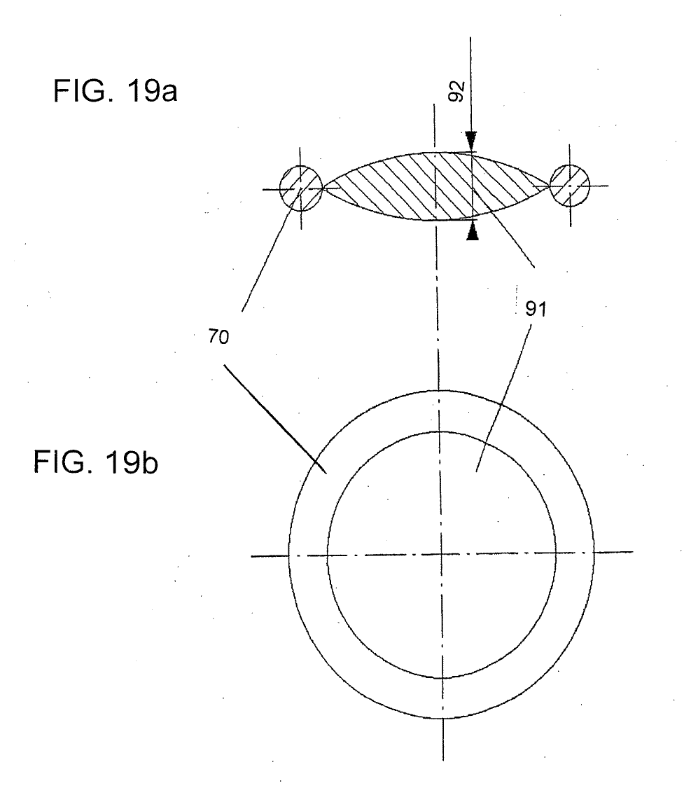

FIG. 19 shows an annular cornea implant with a central implantation body 91 according to the invention.

When correcting farsightedness, the central corneal radius needs to be reduced. This is why a central implantation body (lens) 91 needs to be chosen where the central thickness is higher than the peripheral thickness, whereas the aforementioned criteria related to the material of an annular cornea implant without a central implantation body invariably also apply in this case. In particular, a cornea implant is recommended which is at least partly made of a material with a shape memory or one that can be fine-tuned.

In this case, a central, possibly deformable lens 91, preferably one made of an elastic, transparent, biocompatible material, is enclosed by a ring body 70 with a shape memory. Because of the central lens and the surrounding ring being connected with each other, the lens may be inserted into the cornea pocket together with the ring and unfolded inside the pocket with the help of the ring, according to the invention. This may, for example, be achieved through elastic forces or through the application of a certain temperature or an electric voltage or an electric or magnetic field. The central lens 91 and the ring 70 may be connected with each other in any way, such as by being welded together, by being wrapped up together in the same foil, by being glued together, by melting the ring into the lens, or by integrating the ring into the lens in any way. The central lens body 91 may, for instance, consist of hydrogel, HEMA, polyethylene, or any other polymer or non-polymer plastic. It is essential that the lens body is sufficiently permeable to oxygen and/or nutrients. Moreover, it can be elastic or non-elastic. The refractive index may be of any dimension whatsoever. The lens body 91 itself may also contain a shape memory. Embodiments as for an annular cornea implant without a central lens body 91 also apply without restrictions to annular cornea implants with a central lens body 91.

The ring body 70 may, in particular, be produced from any of the materials described above, including materials where the shape memory can be thermally, electrically or magnetically activated. This allows, for example, to use an electrically or magnetically readjustable ring body (as described elsewhere herein) for changing the tension and thus the central thickness (mean thickness) 92 of the elastic lens body 21, thereby influencing the impact of the latter in correcting the refractive error of the eye. By choosing an appropriate geometry for the central lens body 91, such as a diffractive or refractive bifocal or multifocal lens, also age-related farsightedness (presbyopia) may be corrected. It may, for instance, be designed as a Fresnel lens body. It may consist of a partly or fully non-transparent material. It may also be designed as a lens body that is not dioptrically effective (e.g. without a central thickening 22). Moreover, if made of a non-transparent (e.g. black) material, it may be effective by producing an image through a single slot or multiple slots, or a single hole or multiple holes (i.e. holes that are arranged in a certain way to achieve a diffractive effect). These holes, due to their arrangement, may have a diffractive effect or, in case of only one hole, a stenopeic effect. However, these holes need not be physical holes, but may also be transparent spots in an otherwise non-transparent medium to achieve this effect. The statements made, in particular those relating to (open) ring-shaped structures or segments, not only apply on the basis of the implantation pocket described herein, but also have validity with respect to other implantation pockets, especially for complete or segmented annular pockets, such as those used for implanting Intacs.

For the correction of astigmatism, rings (open, closed, split or segmented) or central bodies with an asymmetrical shape or cross section are needed. Myopic astigmatism is therefore most easily corrected by using a round implant with different cross sections along the main axes, or with a homogeneous cross section but preferably elliptic ring shape, or a combination of both. The same applies to hyperopic astigmatism, with the difference that not the ring but the central body (lens) is asymmetrical, and that this asymmetry is preferably impressed on the body by one of the aforementioned materials and/or shapes. In particular, the central radii of the two main sections of the astigmatic implantation body are different.

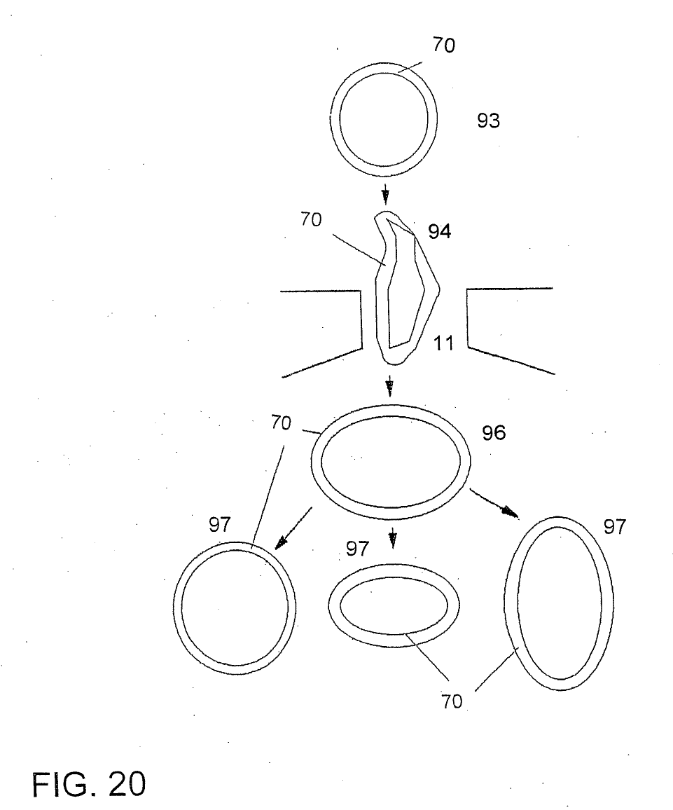

FIG. 20 illustrates the implantation procedure of an annular cornea implant, according to the invention, being inserted into a cornea pocket via a narrow tunnel. To this end, an annular cornea implant 70 with any type of starting shape 93, but preferably one that corresponds to the end shape 96 or 97, is deformed so as to adopt an intermediate shape 94 for inserting into the cornea pocket via a tunnel 11.

Subsequently, through the shape memory according to the invention which is impressed on the implant 70, the intermediate shape 94 is changed into a predefined shape 96 (end shape) inside the cornea pocket. This shape preferably corresponds to the initial starting shape 93, but may also be different. This change of shape may take place automatically or may be triggered directly or indirectly via an appropriate trigger signal. Such a trigger signal is ideally a temperature increase in the implant exceeding a certain transformation temperature. Subsequently, the implant may, in certain cases, change from an end shape 96 into another end shape 97. This change from one end shape 96 into another end shape 97 is ideally triggered by electric or magnetic signals. The end shape 97 may, in particular, be determined by the electric or magnetic field strength applied or by the electric current through the implant 70.

It needs to be stressed that each feature of any embodiment can be combined with any feature of another embodiment, to obtain a new embodiment.

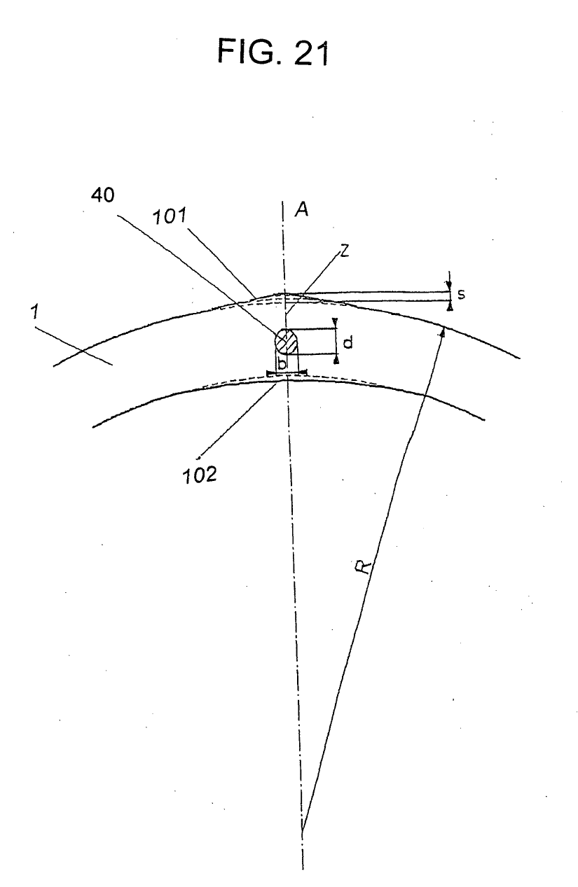

FIG. 21 shows a cross section of the cornea 1 of a human eye with a radius of curvature R including an optical center Z. A corneal implant 40 according to an aspect of the invention is implanted in the corneal tissue of the cornea 1, having an effective thickness d of more than 50 microns, measured in the direction of the optical axis A of the eye, and a width b of less than 1 mm, measured in a plane perpendicular to the direction of thickness.

The corneal implant 40 has no imaging function in relation to the human eye, which means that the light rays entering the eye are not focused on the retina (not depicted in the drawings) of the eye due to the optical properties of the corneal implant 40 according to an aspect of the invention. Instead, the implantation of the corneal implant 40 results in a central volume addition and thus in an aspherical surface contour 101 of the cornea 1 around the optical center Z of the cornea, which also facilitates multi-focal imaging.

In contrast to the known state-of-the-art corneal implants and vision correction methods, an aim of an aspect of the present invention is to introduce a corneal implant 40 into the optical center Z of the eye which deliberately lacks an optical function and which serves to correct the impaired vision exclusively by altering the curvature R of the cornea 1 around the corneal implant.

While this also leads to deformations in the area of the corneal back face 102 these are of only minor relevance for vision correction.

In keeping with the invention, the corneal implant 40 may be of any type of transparency: it may be fully opaque, semi-transparent, or fully transparent. Based on the fact that the corneal implant 40 has no imaging function in relation to the eye, it may be of any color whatsoever, preferably black to assure compatibility with the black pupil.

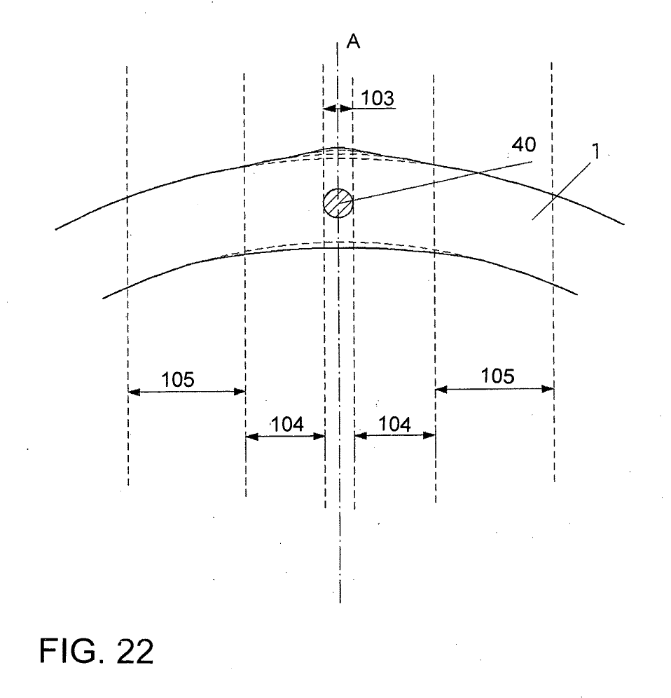

FIG. 22 shows a cross section through the cornea 1 of a human eye in which a corneal implant 40 according to an aspect of the invention is inserted, including markings of the different effective areas of the cornea 1. The central corneal area 103, which is defined by the size of the implant 40, is not directly involved in the visual process. The peripherally adjoining area 104 shows the aspherical surface contour with the resulting property of multi-focal imaging. Then comes the corneal area 105 that remains unaffected by the corneal implant 40.

Depending on the dimensions of the corneal implant 40, there is the possibility to add refractive power for near vision to the area 104, whereas area 105 is intended for far vision. The latter is peripherally confined by the pupil diameter.

The embodiment according to FIGS. 21 and 22 presents the use of a corneal implant 40 which is rotation-symmetrically arranged around the axis of the effective thickness, thus having the shape of a sphere and being limited to 1 mm diameter in size. As far as the aspherical surface contour 101 of the cornea 1 and the multi-focal imaging properties are concerned, such a sphere-shaped corneal implant 40 produces exceptionally positive results.

To the expert it is clear, however, that a corneal implant 40 according to an aspect of the invention may basically have any shape and yet be capable of solving the task underlying the invention, provided the implant has a minimum effective thickness of 50 microns, measured in the direction of the optical axis A of the eye, and a maximum width of 1 mm, measured in a plane perpendicular to the direction of thickness.

It is important to note that due to the introduction of a corneal implant 40 into the optical center of the cornea 1, the curvature R of the cornea around the optical center changes significantly.

The ratio between the width and the effective thickness of the corneal implant should ideally not exceed factor 3 and/or fall below factor 1 to assure acceptable multi-focal imaging properties for the patient. Another requirement is that the width alongside the circumference must not vary by more than 30 percent of the largest width.

FIG. 23 shows a cross section through a corneal implant 40 according to the invention, which has an elliptic cross section with an effective thickness d and a width b.

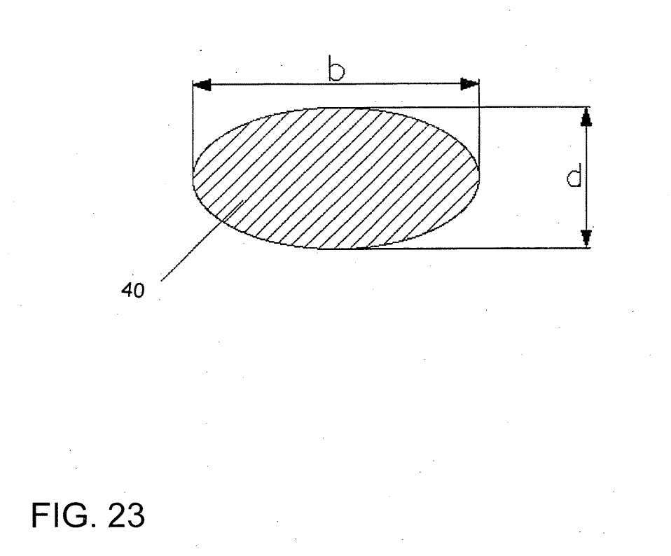

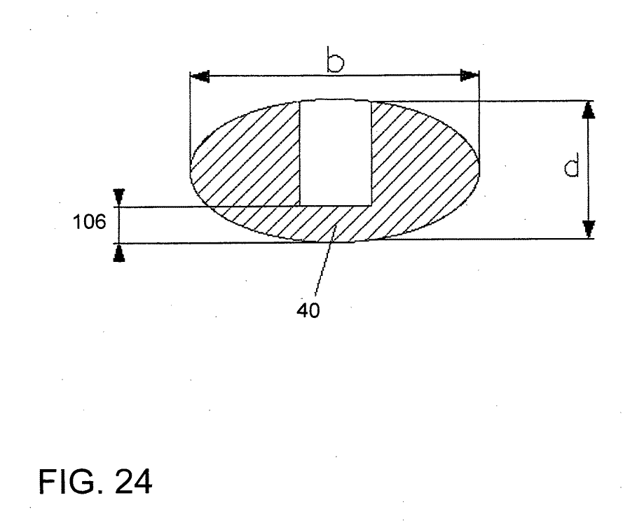

FIG. 24 shows a cross section of an alternative corneal implant 40 according to the invention, with which the same effect in keeping with the invention may be achieved as with the corneal implant 40 depicted in FIG. 23, as long as a minimum effective thickness of 50 microns and a maximum width of 1 mm as indicated above are observed. The expert immediately notices that while cavities, such as those schematically represented in FIG. 24, reduce the thickness of the corneal implant 40 to a minimum thickness 106 in some sections, the effective thickness d remains unaffected therefrom and thus the effect to be achieved according to the invention is not impacted. Consequently, a corneal implant 40 as depicted in FIG. 24 allows for the changing of the curvature R of the cornea 1 in the optical center of the cornea 1, while at the same time leaving the vision unimpaired.

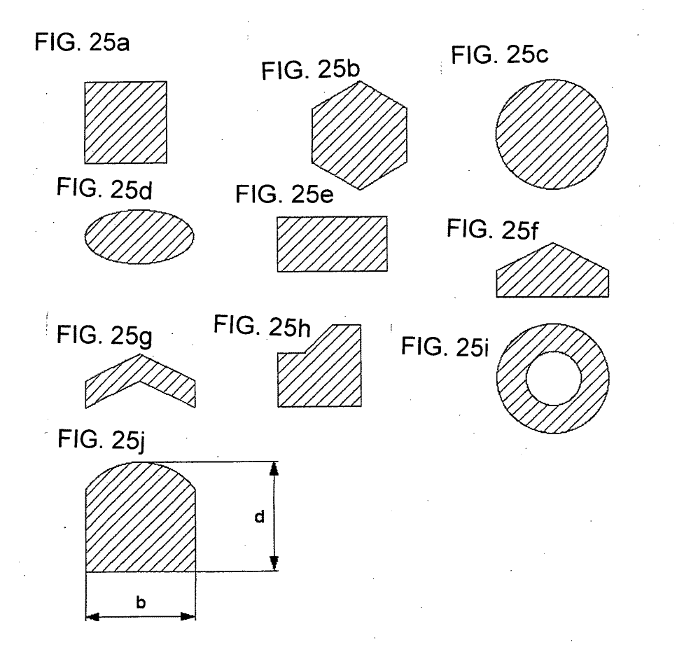

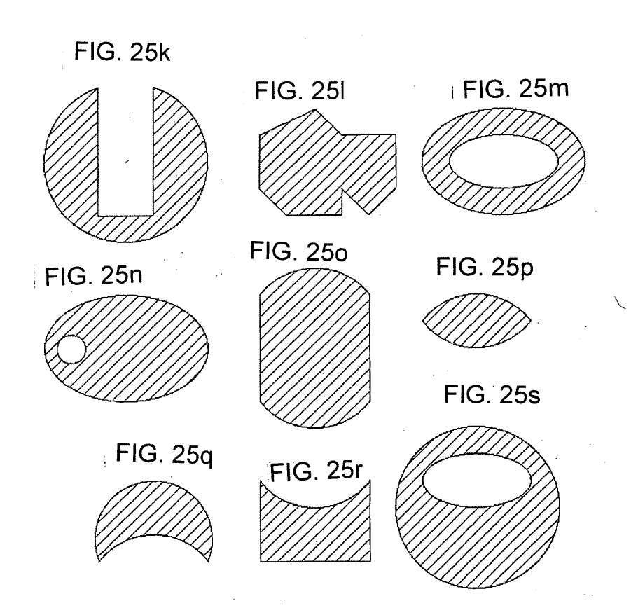

FIGS. 25a to 25s show further potential cross sections of corneal implants 40 according to aspects of the invention which, provided that they fulfill the requirements regarding a minimum effective thickness and a maximum permissible width, support the aspherical surface contour 101 of the cornea 1 required for multi-focal vision.

An aspect of the invention is based on the assumption that a deflection of the cornea 1 is required in order to achieve the desired effect, i.e. the outside measurements of the corneal implant 40 as well as its elasticity need to be adjusted to the elasticity of the cornea 1 and the compression inside the tissue in such a way that the desired deflection and the related aspherical surface contour 101 is achieved. This can be accomplished by using a corneal implant with an effective thickness of more than 50 microns; by limiting the width to below 1 mm, a relevant vision impairment and a nutrition deficiency of the cornea can be prevented and the implantation into the optical center can be achieved.

The diameter and/or the thickness of the implant should be below 1 mm. In particular, since the thickness of the cornea in the center is about 0.5 to 0.6 mm, the thickness and/or the diameter of the implant should be below 0.5 mm, or 0.4 mm or 0.3 mm. Ideally, the thickness and/or diameter should be between 200 and 400 microns.

The material used for the implant may be any type of biocompatible material such as PMMA, HEMA, acryl-containing materials, plastics, metals, semi-conductors, insulators, or other materials commonly used in this field of application.

The method for producing an implant bed may differ from known techniques or may be according to known techniques. The implant bed may, for example, be produced by using a LASIK keratome, by cutting a largely enclosed corneal pocket, as described in EP 1 620 049 B1, or by creating a narrow corneal tunnel.

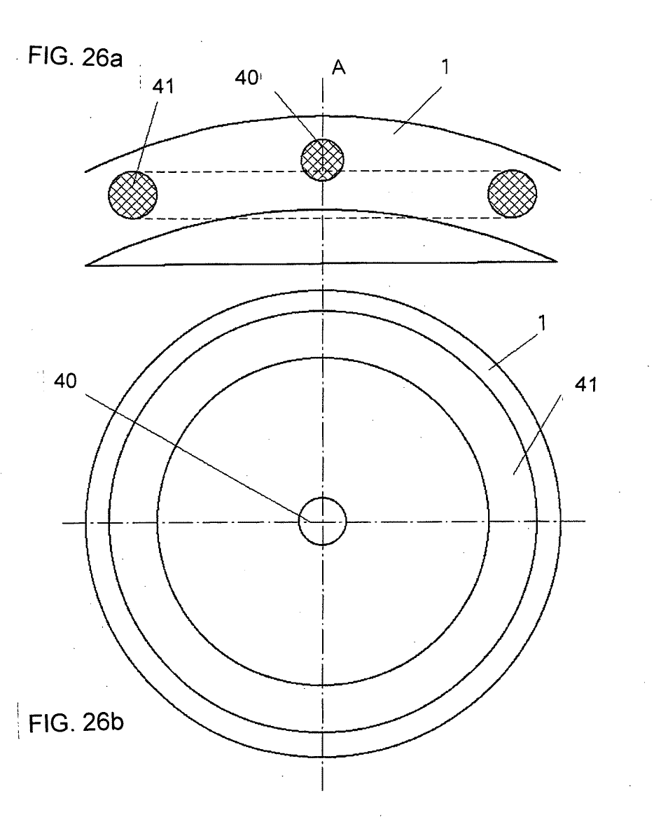

The corneal implant 40 according to an aspect of the invention primarily serves to correct a presbyopic condition, but also presbyopia in combination with hypermetropia. It may also be introduced in combination with other known corneal implants, for instance in combination with ring implants as shown in FIGS. 26a and 26b, by which presbyopia in combination with myopia can be corrected.

FIGS. 26a and 26bb show a known ring-shaped corneal implant 41 which is implanted outside the optical center of the cornea 1, as well as a corneal implant 40 according to an aspect of the invention which is implanted in the optical center of the cornea 1, producing the aspherical surface contour 101. Instead of a ring-shaped corneal implant 41, also individual small implants 42, as shown in FIGS. 7a and 7b, may be applied outside the optical center of the cornea 1.

The corneal implants 40 and 41 or 42 together serve to correct presbyopia in combination with myopia due to the change of curvature of the corneal surface, even though the implants have no optical effect.

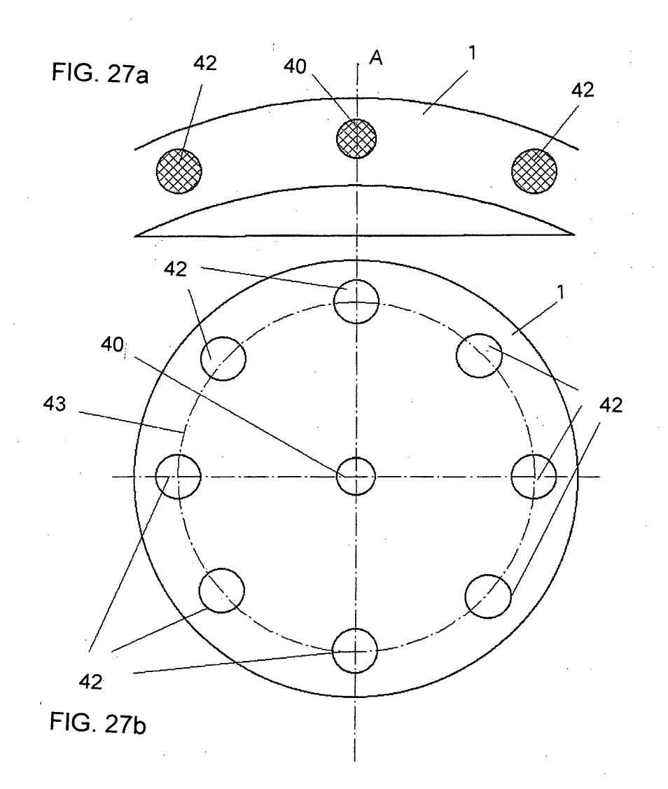

In FIGS. 27a and 27b, the small implants 42 are arranged along a circular line 43 around the optical center. If the small implants 42 have different sizes, in addition to presbyopia and myopia also astigmatism may be corrected.

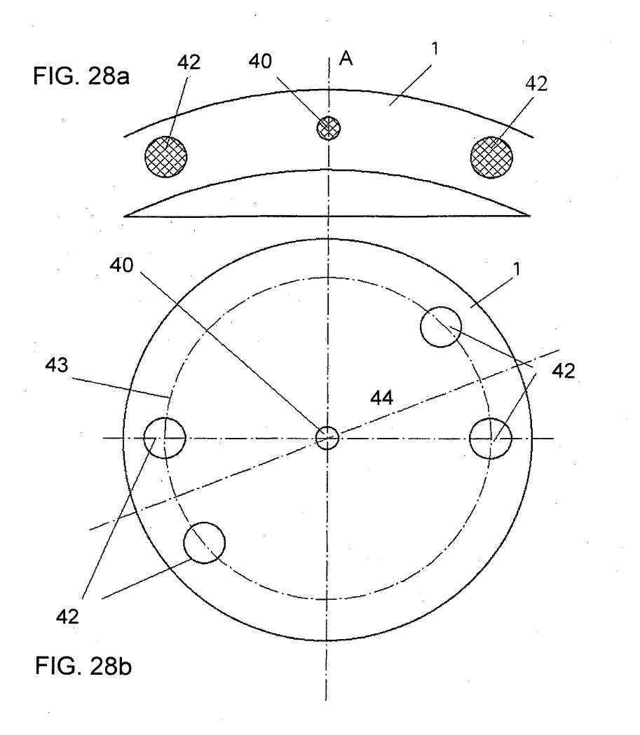

If the corneal implants 40 and 42 are arranged as illustrated in FIGS. 28a and 28b so that a preferred axis 44 is created, also astigmatic vision may be corrected.

Due to the complex curvature of the corneal surfaces in FIGS. 26a-b, 27a-b and 28a-b, the surface is only schematically represented, without the curvature being changed by the corneal implants 40, 41 and 42.

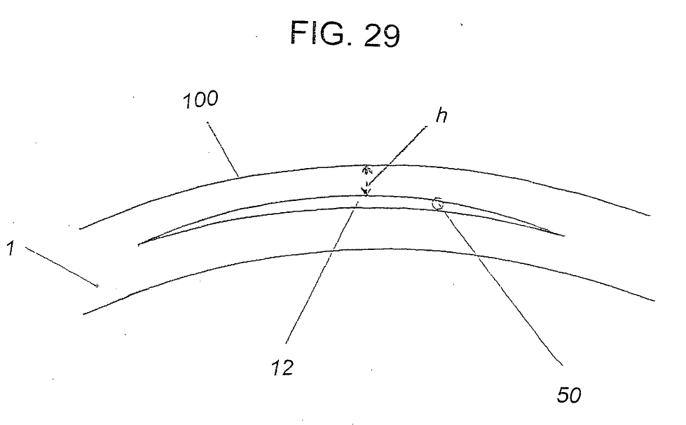

In a further aspect of the invention, a method is provided for the treatment of corneal diseases, such as for example, Keratoconus. As illustrated in FIG. 29, a method for treating corneal disease according to an aspect of the invention includes the step of forming a corneal pocket 12 in a cornea 1 at a depth h from a corneal surface 100.

Corneal pocket 12 may be any suitable shape and/or size and is preferably a complete or incomplete laminar dissection which is roughly or virtually parallel to the front or back surface of the cornea 1. The corneal pocket 12 should be centered at least roughly with respect to the optical or anatomical axis. Alternatively, the corneal pocket 12 may be positioned off-center.

The depth of the corneal pocket 12 measured from the corneal surface 100 should be at least approximately fifty microns and less than approximately four hundred fifty microns. Thus, in a method according to an aspect of the invention, the corneal pocket 12 may be formed at a depth of between approximately fifty microns and four hundred fifty microns from the surface 100 of the cornea 1. In particular, corneal pocket 12 may be formed at a depth of between approximately two hundred fifty microns and three hundred fifty microns from the corneal surface 100. More particularly, corneal pocket 12 may be formed at a depth of approximately three hundred microns from the corneal surface 100.

Corneal pocket 12 is formed to have a sufficient diameter or extension D. The diameter D of the corneal pocket 12 should be less than approximately ten millimeters and larger than approximately six millimeters, preferably more than approximately seven millimeters and more preferably more than approximately eight millimeters. For example, the corneal pocket 12 may have a diameter D of approximately nine millimeters. In other embodiments, the corneal pocket 12 may have a diameter even smaller than approximately six millimeters, and in particular even less than approximately two millimeters. For example, in a method according to an aspect of the invention, corneal pocket 12 may be formed to have a diameter D of between approximately two millimeters and ten millimeters.

The corneal pocket 12 can be a closed pocket or a pocket with an opening which may be very small, for example an incision, or an opening which extends over several clock hours. Preferably, the corneal pocket 12 is closed along its entire circumference with the optional exception of a narrow tunnel-like entry or small pocket entry.

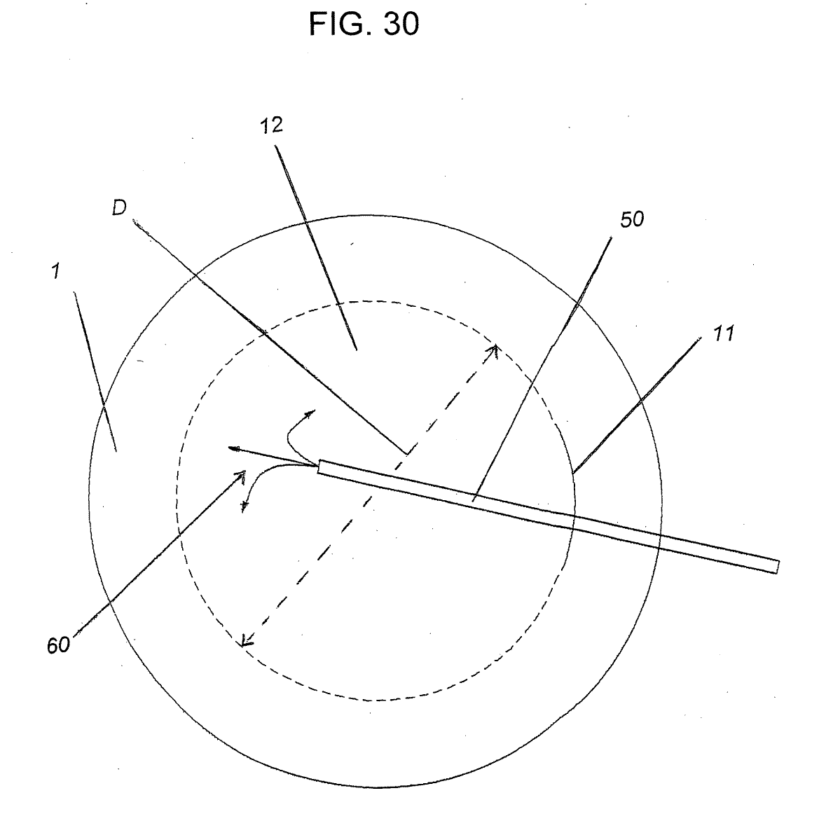

As illustrated, for example in FIGS. 30 and 3a-3d, the step of forming a corneal pocket 12 may include forming a tunnel-like entry or small pocket entry 11 through the corneal surface. The tunnel-like entry 11 may be narrow in width. For example, the width W of the tunnel-like entry 11 may be less than approximately six millimeters, preferably less than approximately five millimeters and more preferably less than approximately four millimeters. The entry opening of the corneal pocket 12 may, however, be of any other suitable size.

Formation of the corneal pocket 12 may be accomplished using a number of techniques and devices. For example, a mechanical microkeratome may be used to form the corneal pocket 12. Alternatively, laser cutting (using, for example a Femtosecond laser) may be used for form the corneal pocket 12. Manual dissection using a suitable manual dissector (such as, for example a crescent knife) is also possible; however, this technique has a much higher degree of difficulty and level of risk.

Exemplary devices suitable for forming a corneal pocket 12 in a method according to an aspect of the invention are described, for example, above and in applicant's co-pending U.S. patent application Ser. No. 10/555,353, the disclosure of which is hereby incorporated by reference in its entirety.

For example, FIGS. 1 and 2 show exemplary devices for forming a corneal pocket 12 in a cornea 1 in a method according to an aspect of the invention.

As illustrated in FIG. 30, a method for treating corneal disease according to an aspect of the invention further includes the step of introducing or instilling a corneal stiffening agent or substance 60 into the corneal pocket 12. The introduction of the corneal stiffening substance 60 into the corneal pocket 12 may be performed via an appropriate cannula 50 and a syringe.

The corneal stiffening substance 60 supports the stiffening of corneal tissue and as a result stops the progression of the diseases in question. For example, the corneal stiffening substance 60 may promote cross-linking of the corneal tissue. The corneal stiffening substance may also have the ability to change the shape of the cornea immediately or over time.

The corneal stiffening substance or agent 60 may be a solid or a gel, but preferably is a liquid. The corneal stiffening substance 60 may be introduced into the corneal pocket 12 through the tunnel-like entry or incision 11, although it is also possible to inject the corneal stiffening substance 60 by means of a cannula via the walls of the corneal pocket 12.

The corneal stiffening substance 60 can be a pure or diluted substance. The amount of corneal stiffening substance applied to the cornea may be between approximately 0.1 milliliters and 10 milliliters, and is preferably approximately 3 milliliters. Penetration of the corneal stiffening substance 60 into the corneal tissue may be achieved either by rinsing, flushing or irrigating the corneal pocket 12 over a limited period of time with the corneal stiffening substance 60. Another way of administering the corneal stiffening substance 60 is to create a depot of the corneal stiffening substance 60 within the corneal pocket 12 over a limited period of time. The associated period of time for applying or administering the corneal stiffening substance 60 via the corneal pocket may be thirty minutes or less, is preferably less than fifteen minutes, and is more preferably less than five minutes. For example, in a method according to an aspect of the invention, the corneal stiffening substance 60 may be applied or administered over a time period of approximately two to three minutes.

One exemplary corneal stiffening substance 60 for introduction into the corneal pocket 12 is riboflavin. For example, in a method according to an aspect of the invention, the corneal stiffening substance 60 includes 0.1% riboflavin diluted in a 20% dextran 500 solution. In general, the corneal stiffening substance 60 may be hypotonic, isotonic or hypertonic or hypoosmolar, isoosmolar or hyperosmolar and can be combined with any suitable diluent. The active substance which may be diluted or pure may be any suitable corneal stiffening substance or composition of substances.

A method for treating corneal disease according town aspect of the invention further includes the step of irradiating the cornea with electromagnetic radiation. The electromagnetic radiation may comprise, for example ultraviolet (UV) light and more particularly may comprise ultraviolet-A (UV-A) light. For example, the irradiation of the cornea may be performed using a UV-A light source providing an irradiation of the cornea at approximately 340 to 380 nanometers wavelength (such as, for example, 365 nanometers) with an intensity between approximately 0.1 milliwatts/square centimeter and 20 milliwatts/square centimeter (such as, for example, 3 milliwatts/square centimeter) over a limited area at the corneal surface between approximately 3 square millimeters and 120 square millimeters, preferably between approximately 70 square millimeters and 100 square millimeters, corresponding roughly to an irradiated diameter at the corneal surface of approximately 9 to 11 millimeters. The irradiated zone may be centered or off-center.

The duration of the irradiation of the cornea may be less than one hour. In particular, in a method according to an aspect of the invention, the cornea is irradiated with electromagnetic radiation for less than thirty minutes and more particularly for less than fifteen minutes.

The irradiation of the cornea with electromagnetic radiation should preferably be performed after the introduction of the corneal stiffening substance 60 into the corneal pocket 12 as described above. In a method according to an aspect of the invention, the step of irradiating the cornea 1 with electromagnetic radiation is performed without removing the epithelium of the cornea. In particular, the irradiation of the cornea 1 may be performed without removing or substantially altering the epithelium either before or during the irradiation process.

A method for treating corneal disease according town aspect of the invention may optionally includes the further step of inserting a corneal implant 40, 41, 42, 70 into the corneal pocket 12. In particular, the corneal implant 40, 41, 42, 70 may be inserted into the corneal pocket 12 through the tunnel-like entry 11.

The corneal implant may be any implant suitable to correct a refractive error. For example, the corneal implant may be a continuous ring implant as illustrated in FIGS. 17a-17d or a split ring implant, as illustrated in FIGS. 18a-18d. The corneal implant may comprise a compressible implant.

Exemplary corneal ring implants suitable for inserting into a corneal pocket in a method according to an aspect of the invention and methods of their use are described, for example, above and in applicant's co-pending U.S. patent application Ser. No. 12/224,966, the disclosure of which is hereby incorporated by reference in its entirety.

Additional exemplary corneal implants suitable for inserting into a corneal pocket in a method according to an aspect of the invention and methods of their use are described, for example, above and in applicant's co-pending U.S. patent application Ser. No. 12/227,533, the disclosure of which is hereby incorporated by reference in its entirety.

For example, FIG. 21 shows a cross-section of a cornea 1 with a corneal implant 40 for insertion into a corneal pocket 12 in a method according to an aspect of the invention. In particular, FIG. 21 shows a cross section of the cornea 1 of a human eye with a radius of curvature R including an optical center Z. A corneal implant 40 is implanted in the corneal tissue of the cornea 1, having an effective thickness d of more than 50 microns, measured in the direction of the optical axis A of the eye, and a width b of less than 1 millimeters, measured in a plane perpendicular to the direction of thickness. The implant 40 may also be placed off-center.

Corneal implant 40 has no imaging function in relation to the human eye, which means that the light rays entering the eye are not focused on the retina (not depicted in the drawings) of the eye due to the optical properties of the corneal implant 40. Instead, the implantation of the corneal implant 40 results in a central volume addition and thus in an aspherical surface contour 101 of the cornea 1 around the optical center Z of the cornea 1, which also facilitates multi-focal imaging. In a particular embodiment, the implant 40 may have an optical function, for example, as a lens or pinhole.

In contrast to the known state-of-the-art corneal implants and vision correction methods, corneal implant 40 deliberately lacks an optical function and may be introduced into the optical center Z of the eye. In particular, corneal implant 40 serves to correct the impaired vision exclusively by altering the curvature R of the cornea 1 around the corneal implant 40. Although this arrangement may lead to deformations in the area of the corneal back face 102, these are of only minor relevance for vision correction.

Corneal implant 40 may be of any type of transparency; for example it may be fully opaque, semi-transparent, or fully transparent. Moreover, because corneal implant 40 has no imaging function in relation to the eye, it may be of any color whatsoever, preferably black to assure compatibility with the black pupil.

In a method according to an aspect of the invention, the step of inserting the corneal implant 40, 41, 42, 70 may be performed after the step of creating the corneal pocket 12, after the step of introducing the corneal stiffening substance 60 into the corneal pocket 12 and after the step of irradiating the cornea 1 with electromagnetic radiation. Thus, the step of inserting the corneal implant 40, 41, 42, 70 may be performed as a fourth step (i.e., after creation of the corneal pocket 12, after introduction of the corneal stiffening substance 60 into the corneal pocket 12 and after irradiation of the cornea 1).

In a method according to another aspect of the invention, the step of inserting the corneal implant 40, 41, 42, 70 may be performed after the step of creating the corneal pocket 12, after the step of introducing the corneal stiffening substance 60 into the corneal pocket 12 and before the step of irradiating the cornea 1 with electromagnetic radiation. Thus, the step of inserting the corneal implant 40, 41, 42, 70 may be performed as a third step (i.e., after creation of the corneal pocket 12, after introduction of the corneal stiffening substance 60 into the corneal pocket 12 and before irradiation of the cornea 1).

In a method according to another aspect of the invention, the step of inserting the corneal implant 40. 41, 42, 70 may be performed after the step of creating the corneal pocket 12, before the step of introducing the corneal stiffening substance 60 into the corneal pocket 12 and before the step of irradiating the cornea 1 with electromagnetic radiation. Thus, the step of inserting the corneal implant 40, 41, 42, 70 may be performed as a second step (i.e., after creation of the corneal pocket 12, before introduction of the corneal stiffening substance 60 into the corneal pocket 12 and before irradiation of the cornea 1).

In this embodiment, the creation of a depot of the cornea stiffening substance 60 within the corneal pocket 12 over a limited period of time is very easy if a continuous ring implant is inserted and if the corneal stiffening substance 60 is introduced “inside the ring” and the depot is delimited by the ring implant. In this case, the anterior and posterior lamellae of the cornea (for example, the anterior and posterior wall of the corneal pocket) as well as the ring implant itself capture or delimit the depot of corneal stiffening substance 60 in the pocket and prevents the corneal stiffening substance 60 from leaking (for example, flowing via the entry to outside the corneal tissue instead of diffusing into the tissue via the pocket walls).

In a method according to an aspect of the invention, a suitable corneal implant may be either centered on the optical axis or the anatomical axis of the cornea of the eye, or alternatively positioned off-center with respect to the optical axis or the anatomical axis of the cornea of the eye.

According to another aspect of the invention, a method is provided for treating refractive errors and vision disorders of an eye. The method includes fixing a suction ring to an eye to be treated. Exemplary devices capable of fixing a suction ring to an eye according to an aspect of the invention are described, for example, above and in applicant's co-pending U.S. patent application Ser. No. 10/555,353, the disclosure of which is hereby incorporated by reference in its entirety.

For example, FIGS. 1 and 2 show exemplary devices capable of fixing a suction ring or fixation ring 5 an eye according to an aspect of the invention.

A method according to an aspect of the invention further includes the step of applanating a cornea of the eye to be treated. Applanating refers to the flattening of the cornea, for example by application of a vacuum. Exemplary devices capable of applanating a cornea according to an aspect of the invention are described, for example, above and in applicant's co-pending U.S. patent application Ser. No. 10/555,353, the disclosure of which is hereby incorporated by reference in its entirety.

For example, FIGS. 1, 2, 7, 8a-g and 10 show exemplary devices including applanators 7 for applanting the cornea.

A method according to an aspect of the invention further includes the step of guiding a blade into the cornea with a guiding mechanism. Exemplary devices capable of guiding a blade into the cornea according to an aspect of the invention are described, for example, above and in applicant's co-pending U.S. patent application Ser. No. 10/555,353, the disclosure of which is hereby incorporated by reference in its entirety.

For example, FIGS. 1, 2 and 5 show exemplary devices capable of guiding a blade 4 into a cornea with a guiding mechanism, for example holding device 3, according to an aspect of the invention.

A method according to an aspect of the invention further includes the step of creating a pocket inside the cornea by guiding the blade inside the cornea. Exemplary devices and techniques for creating a pocket inside the cornea according to an aspect of the invention are described, for example, above and in applicant's co-pending U.S. patent application Ser. Nos. 10/555,353 and 12/925,162, the disclosures of which are hereby incorporated by reference in its entirety.

For example, FIGS. 1 and 2 show exemplary devices capable of creating a pocket 12 inside the cornea 1 by guiding the blade 4 inside the cornea 1 according to an aspect of the invention. Moreover, FIGS. 3a-3d and 4a-4b show an exemplary techniques for creating a pocket 12 in a cornea 1 by guiding the blade 4 inside the cornea 1.

A method according to an aspect of the invention further includes the steps of removing the blade from the cornea and removing the suction ring from the eye.

In a further aspect of the invention, the blade is provided with a pointed tip. For example, FIG. 9 shows an exemplary blade 4 suitable for treating refractive errors and vision disorders according to an aspect of the invention. As illustrated in FIG. 9, the blade 4 may have a pointed tip 19 with two edges 20 originating from this tip and may resemble a double-edged knife. The blade may be a non-metallic blade.

In a further aspect, the method includes the step of connecting the suction ring and the blade with the guiding mechanism. Exemplary devices wherein the suction ring and blade are connected with a guiding mechanism according to an aspect of the invention are described, for example, above and in applicant's co-pending U.S. patent application Ser. No. 10/555,353, the disclosure of which is hereby incorporated by reference in its entirety.

For example, FIGS. 1 and 2 show exemplary devices wherein suction ring 5 and blade 4 are connected with a guiding mechanism or holding device 3.

In a further aspect of the invention, the blade is vibrated as it is guided into the cornea with the guiding mechanism and/or as it is guided inside the cornea to create the pocket inside the cornea. Exemplary devices adapted for vibrating a blade as the blade is guided into the cornea and/or guided inside the cornea to create a pocket are described, for example, above and in applicant's co-pending U.S. patent application Ser. No. 10/555,353, the disclosure of which is hereby incorporated by reference in its entirety.

For example, FIG. 5 shows an exemplary device including a vibrator 14 for setting blade 4 in oscillatory motion. The vibrating element for vibrating the blade may include, for example a piezo element or an unbalanced motor.

In a further aspect of a method according to the invention, the blade does not touch the suction ring or the guiding mechanism as the cornea is being cut. For example as described above and shown in the exemplary devices illustrated in FIGS. 1 and 2, the blade 4 may pass through a frame recess 10 with clearance, such that it does not rest on the frame. In this way, the blade may be prevented from touching the suction ring or guiding mechanism as the cornea 1 is being cut.

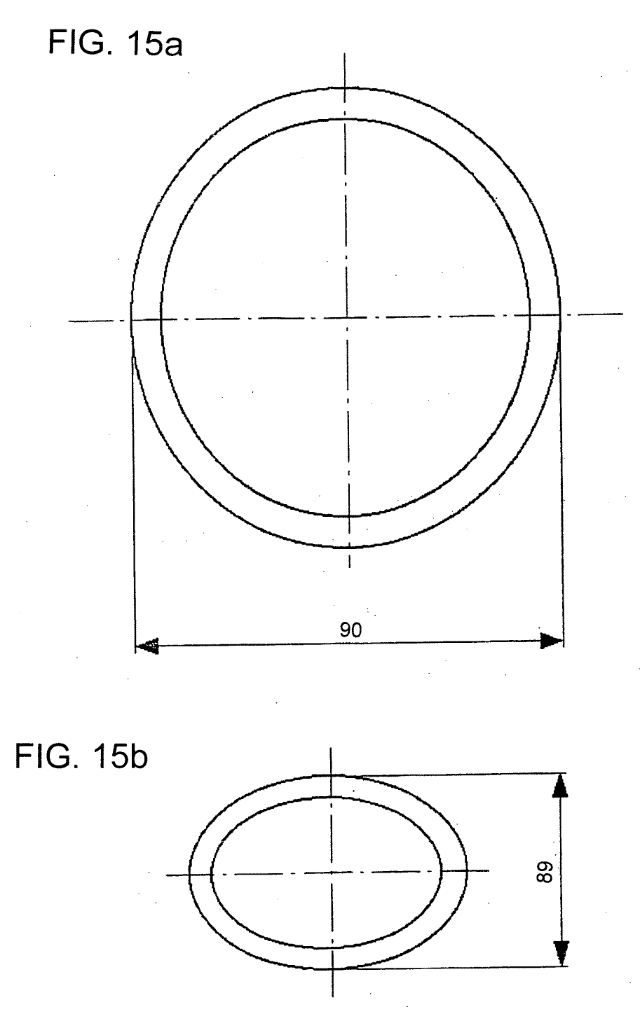

In a further aspect of a method according to the invention, an implant or inlay may be inserted into the pocket inside the cornea. For example, various corneal implants which may be inserted into the pocket in the cornea according to aspect of the invention are described above and illustrated in FIGS. 11, 12, 13, 14, 15a-b, 16a-b, 17a-d, 18a-d, 19a-b, 20, 21, 22, 23, 24, 25a-s, 26a-b, 27a-b and 28a-b.

The implant or inlay may have a ring shape, such as a continuous or closed ring shape, as illustrated for example in FIGS. 17a-d or a non-continuous or split ring shape, as illustrated for example in FIGS. 18a-d.

The ring-shaped implant or inlay may be compressible and may have a shape memory. Exemplary shape memory materials suitable for an implant according to an aspect of the invention are described above and include PMMA, polymers of EEMA or HEMA, or other acrylic materials, hydrogels, nylon, silicon, polycarbonate, polyethylene or other plastics, plastics with a temperature-dependent shape memory, shape memory alloys (e.g. based on Ni—Ti, Cu—Zn—Al, Cu—Al—Ni, etc.), suitable compounds of plastics and metals or non-metals (e.g. ceramics, semi-conductors, etc.), suitable compounds of metals and non-metals (e.g. ceramics, semi-conductors, etc.), or composite materials.

The shape memory characteristics of the implant or inlay may result from the geometry and/or the material used. Suitable materials may be elastically or non-elastically deformable (plastic) materials. In elastic materials with a shape memory, the deformability and adjustment force mainly results from the elasticity of the material, such as PMMA (polymethyl methacrylate), silicone, etc. In non-elastic materials such as shape memory alloys, the adjustment force results, for example, from atomic forces which are released when one grid structure is spontaneously transformed into another.

Moreover, the shape memory of the material used to form the implant may be activated, for example by electrical fields, mechanical forces (e.g. by using ultrasound or reducing frictional forces inside the pocket), thermal conditions, chemical conditions (e.g. pH values), and/or magnetic fields.

Typical materials with a shape memory that can be activated are for instance, polymer metals, ionic polymer-metal composites, IMPC, electroactive polymers (e.g. electronic or ionic EAP materials) such as polyacrylonitril (PAN), ceramics or electroactive ceramics, suited conductor or semi-conductor plastics, ionic polymeric conductor composites, IPCC, and magnetic shape memory elements such as those based on NiMnGa or Ni2MnGa.

Shape memory alloys may be alloys based on Ni—Ti, Cu—Zn—Al, Cu—Al—Ni and other materials. Plastics with a temperature-dependent shape memory as well as shape memory alloys may be easily deformed (in particular also by plastic deformation) preferably below a specific transformation temperature, but resume a predefined shape above this transition temperature. In this manner, changing the temperature of the folded cornea implant in its intermediate shape may help to achieve its unfolding into the desired shape after being inserted into the stromal implantation bed.

Materials with a shape memory may further include materials which change size, elasticity or plasticity in the wake of swelling or de-swelling, such as by absorption or extraction of water. Such materials may include completely or incompletely hydrated plastics, such as HEMA or hydrogels.

In a further aspect of a method according to the invention, the implant or inlay inserted into the pocket may no dimensions larger than 1 millimeter.

In a further aspect of a method according to the invention, the implant or inlay inserted into the pocket may have no direct optical or imaging function. For example, the implant or inlay may comprise a corneal implant introduced into the optical center of the eye which lacks an optical function and which serves to correct impaired vision by altering the curvature of the cornea around the corneal implant. Such implants are described above.

In a further aspect of a method according to the invention, the implant or inlay may be positioned inside the pocket inside the cornea. The implant or inlay may be positioned in a center of the cornea and/or a center of a visual axis.

In a further aspect of the invention, the method for treating refractive errors and vision disorders of the eye may include the step of introducing a corneal stiffening substance into the pocket inside the cornea. The corneal stiffening substance may be introduced into the pocket using the techniques and devices described above.

For example, FIG. 30 shows the introduction or instillation of a corneal stiffening agent or substance 60 into a corneal pocket 12 according to an aspect of the invention. The introduction of the corneal stiffening substance 60 into the corneal pocket 12 may be performed via an appropriate cannula 50 and a syringe.

The corneal stiffening substance 60 supports the stiffening of corneal tissue and as a result stops the progression of the diseases in question. For example, the corneal stiffening substance 60 may promote cross-linking of the corneal tissue. The corneal stiffening substance may also have the ability to change the shape of the cornea immediately or over time.

The corneal stiffening substance or agent 60 may be a solid or a gel, but preferably is a liquid. The corneal stiffening substance 60 may be introduced into the corneal pocket 12 through the tunnel-like entry or incision 11, although it is also possible to inject the corneal stiffening substance 60 by means of a cannula via the walls of the corneal pocket 12.

The corneal stiffening substance 60 can be a pure or diluted substance. The amount of corneal stiffening substance applied to the cornea may be between approximately 0.1 milliliters and 10 milliliters, and is preferably approximately 3 milliliters. Penetration of the corneal stiffening substance 60 into the corneal tissue may be achieved either by rinsing, flushing or irrigating the corneal pocket 12 over a limited period of time with the corneal stiffening substance 60. Another way of administering the corneal stiffening substance 60 is to create a depot of the corneal stiffening substance 60 within the corneal pocket 12 over a limited period of time. The associated period of time for applying or administering the corneal stiffening substance 60 via the corneal pocket may be thirty minutes or less, is preferably less than fifteen minutes, and is more preferably less than five minutes. For example, in a method according to an aspect of the invention, the corneal stiffening substance 60 may be applied or administered over a time period of approximately two to three minutes.

One exemplary corneal stiffening substance 60 for introduction into the corneal pocket 12 is riboflavin. For example, in a method according to an aspect of the invention, the corneal stiffening substance 60 includes 0.1% riboflavin diluted in a 20% dextran 500 solution. In general, the corneal stiffening substance 60 may be hypotonic, isotonic or hypertonic or hypoosmolar, isoosmolar or hyperosmolar and can be combined with any suitable diluent. The active substance which may be diluted or pure may be any suitable corneal stiffening substance or composition of substances.

In a further aspect of the invention, the method for treating refractive errors and vision disorders of the eye may include the step of irradiating the cornea with electromagnetic radiation. The electromagnetic radiation may be administered using the techniques and devices described above.

For example, the electromagnetic radiation may comprise, ultraviolet (UV) light and more particularly may comprise ultraviolet-A (UV-A) light. The irradiation of the cornea may be performed, for example, using a UV-A light source providing an irradiation of the cornea at approximately 340 to 380 nanometers wavelength (such as, for example, 365 nanometers) with an intensity between approximately 0.1 milliwatts/square centimeter and 20 milliwatts/square centimeter (such as, for example, 3 milliwatts/square centimeter) over a limited area at the corneal surface between approximately 3 square millimeters and 120 square millimeters, preferably between approximately 70 square millimeters and 100 square millimeters, corresponding roughly to an irradiated diameter at the corneal surface of approximately 9 to 11 millimeters. The irradiated zone may be centered or off-center.

The duration of the irradiation of the cornea may be less than one hour. In particular, in a method according to an aspect of the invention, the cornea is irradiated with electromagnetic radiation for less than thirty minutes and more particularly for less than fifteen minutes.

In an aspect of the invention wherein a corneal stiffening substance is inserted into the pocket, the irradiation of the cornea with electromagnetic radiation may be performed after the introduction of the corneal stiffening substance.

Accordingly, while a number of embodiments of the present method have been shown and described, it is obvious that many changes and modifications may be made thereunto without depar-ting from the spirit and scope of the invention.

1. A method for treating refractive errors and vision disorders of an eye, the method comprising the steps of:
a) fixing a suction ring to an eye;
b) applanating a cornea of the eye;
c) guiding a blade into the cornea with a guiding mechanism;
d) creating a pocket inside the cornea by guiding the blade inside the cornea;
e) removing the blade from the cornea; and
f) removing the suction ring from the eye.

2. The method according to claim 1, further comprising the step of providing the blade with a pointed tip.

3. The method according to claim 1, further comprising the step of connecting the suction ring and the blade with the guiding mechanism.

4. The method according to claim 1, wherein the blade is a non-metallic blade.

5. The method according to claim 1, further comprising the step of vibrating the blade as the blade is guided into the cornea with the guiding mechanism.

6. The method according to claim 1, further comprising the step of vibrating the blade as the blade is guided inside the cornea to create the pocket inside the cornea.

7. The method according to claim 1, wherein the blade does not touch the suction ring or the guiding mechanism as the cornea is being cut.

8. The method according to claim 1, further comprising the step of inserting an implant into the pocket inside the cornea.

9. The method according to claim 8, wherein the step of inserting an implant into the pocket inside the cornea comprises inserting an implant having a ring shape.

10. The method according to claim 9, wherein the step of inserting an implant having a ring shape comprises inserting an implant having a continuous ring shape.

11. The method according to claim 9, wherein the step of inserting an implant having a ring shape comprises inserting an implant having a non-continuous ring shape.

12. The method according to claim 9, wherein the step of inserting an implant having a ring shape comprises inserting a compressible implant.

13. The method according to claim 8, wherein the step of inserting an implant into the pocket inside the cornea comprises inserting an implant having a shape memory.

14. The method according to claim 8, wherein the step of inserting an implant into the pocket inside the cornea comprises inserting an implant having no dimensions larger than 1 millimeter.

15. The method according to claim 14, wherein the step of inserting an implant into the pocket inside the cornea comprises inserting an implant having no direct optical function.

16. The method according to claim 1, further comprising the step of positioning an implant inside the pocket inside the cornea.

17. The method according to claim 16, wherein the step of positioning an implant inside the pocket inside the cornea comprises positioning the implant in at least one of a center of the cornea and a center of a visual axis.

18. The method according to claim 1, further comprising the step of introducing a corneal stiffening substance into the pocket inside the cornea.

19. The method according to claim 1, further comprising the step of irradiating the cornea with electromagnetic radiation.
